(19) United States
(12) Patent Application Publication
(10) Pub. No.: US 2007/0122865 A1
(43) Pub. Date: May 31, 2007

(54) Enzymes and enzymic processes

(75) Inventor: Gianfranco Gilardi, London (GB)

Correspondence Address: SUGHRUE MION, PLLC, 2100 PENNSYLVANIA AVENUE, N.W., SUITE 800, WASHINGTON, DC, 20037, US

(73) Assignee: NANOBIODESIGN LIMITED, LONDON E14 2WQ (GB)

(21) Appl. No.: 10/485,985

(22) PCT Filed: Aug. 8, 2002
(86) PCT No.: PCT/GB02/03648

§ 371 Date: Nov. 2, 2004

(30) Foreign Application Priority Data
Aug. 8, 2001 (GB) 0119366.3

(51) Int. Cl. C12Q 1/26; C07H 21/04; C12P 21/06; C12N 9/02; C12N 15/74; C12N 1/21

(52) U.S. Cl. 435/25; 435/69.1; 435/189; 435/252.33; 435/488; 536/23.2

(57) ABSTRACT

A chimeric protein comprises a haem domain from a mammalian or plant cytochrome P450 and a scaffold domain generally comprising an electron transfer domain, from P450 BM3 of Bacillus megaterium. The protein does not include the membrane binding portion of the wild type mammalian or plant P450 and is hence water soluble. The protein is enzymnically active on substrates for the wild-type mammalian or plant P450 and electrons are transferred to electron transfer portions for instance part of the scaffold domain, such as FAD or FMN. The protein is useful for analysing the substrate specificity of the haem domain or for detecting substrates which are analytes of interest.

The present invention relates to chimeric proteins comprising a catalytic domain and a scaffold domain derived from different sources.

Cytochromes P450 (P450) are highly relevant to the bio-analytical area (Sadeghi et al, 2001). They form a large family of enzymes present in all tissues important to the metabolism of most of the drugs used today, playing an important role in the drug development and discovery process (Poulos, 1995, Guengerich, 1999). They catalyse the insertion of one of the two atoms of an oxygen molecule into a variety of substrates (R) with quite broad regioselectivity, resulting in the concomitant reduction of the other oxygen atom to water, according to the reaction:
RH+O₂+2e⁻+2H⁺→ROH+H₂O

Despite their importance, applications in the bio-analytical area are difficult due to problems related to their poor interaction with electrode surfaces and the association to biological membranes of the mammalian P450. Nevertheless, an exciting potential application of these enzymes relies in the creation of electrode arrays for high-through-put screening for propensity to metabolic conversion or toxicity of novel potential drugs.

In order to achieve this goal, this issue needs to be addressed: The ability of handling stable and soluble human P450 enzyme;

Cytochrome P450 BM3 is a soluble, catalytically self-sufficient fatty acid monoxygenase isolated from Bacillus megaterium (Narhi and Fulco, 1986 and 1987). It is particularly interesting in that it has a multi-domain structure, composed of three domains: one FAD one FMN and one haem domain, fused on the same 119 kDa polypetidic chain of 1048 residues. Furthermore, despite its bacterial origin, P450 BM3 has been classified as a class II P450 enzyme, typical of microsomal eukaryotic P450s (Ravichandran et al., 1993): it shares 30% sequence identity with microsomal fatty acid w-hydroxylase, 35% sequence identity with microsomal NADPH:P450 reductase, and only 20% homology with other bacterial P450s (Ravichandran et al., 1993). These characteristics have suggested the use of P450 BM3 as a surrogate for mammalian P450s, and this has been recently substantiated when the structure of rabbit P450 2C5 was solved (Williams et al., 2000).

Mammalian P450 enzymes are membrane bound. As such they are difficult to isolate from their physiological sources, and to use in test systems. Examples of mammalian P450 enzymes (CYP's) are shown in table 1

TABLE 1
Mammalian CYPs and their known functions

| Function | CYP |
|---|---|
| Drug metabolism | CYP1A1, CYP1A2, CYP1B1, CYP2A6, |
| | CYP2A7, CYP2A13, CYP2B6, CYP2C8, |
| | CYP2C18, CYP2C19, CYP2D6, CYP2E1, |
| | CYP2F2, CYP2G1, CYP2J1, CYP2R2, |
| | CYP2S1, CYP3A4, CYP3A5, CYP3A11 |
| Arachidonic acid or fatty | CYP4A11, CYP4B1, CYP4F2, CYP4F3, |
| acid metabolism | CYP4F8, CYP4F11, CYP4F12, CYP4X1, |
| | CYP4Z1 |
| Thromboxane A2 synthase | CYP5A1 |
| Bile acid biosynthesis | CYP7A1, CYP8B1, CYP27A1 |
| Brain specific form of | CYP7B1 |
| 7-alpha hydroxylase | |
| Prostacyclin synthase | CYP8A1 |
| Steroid biosynthesis | CYP11A1, CYP11B1, CYP11B2, CYP17, |
| | CYP19, CYP21A2 |
| Vitamin D degradation | CYP24 |
| Retinoic acid hydroxylase | CYP26A1 |
| (probably) | |
| Retinoic acid hydroxylase | CYP26B1 |
| Vitamin D3 1-alpha | CYP27B1 |
| hydroxylase | |
| (Not known) | CYP39 |
| Cholesterol 24-hydroxylase | CYP46 |
| Cholesterol biosynthesis | CYP51 |

P450 2E1 or (CYP2E1) is a microsomal enzyme present in the liver and other tissues of many mammalian species that has been shown to catalyse the oxidation of over 50 compounds, including benzene, acetone, chloroform, ethanol and other alcohols, a number of N-nitrosamines, small halogenated hydrocarbons and vinyl monomers, drugs such as acetaminophen and chlorzoxazone (Lieber, 1997). Having as substrates ethanol and many suspect carcinogens, P450 2E1 has been considered of great interest for its possible relevance to alcoholism and chemical carcinogenesis (Gillam et al., 1994). Further substrates of P450 2E1 are shown in table 2.

### Table 2

### Substrates of Cytochrome P450 2E1 (Lieber, 1997)

Alcohols, Aldehydes, Ketones and Nitriles

Acetaldehyde, Butanol, 2-butanone, Ethanol, Glycerol, Isopropanol, Methanol, Propanol, Pentanol, 1-Phenylethanol (to acetophenone).

Aromatic Compounds

Acetaminophen (Tylenol), Aniline, Benzene, Bromobenzene, Caffeine (to theophylline and theobromine), Casaicin, Chlorzoxazone (Parafon), 3-Hydroxypyridine, Isoniazid, Phenol, Pyridine, p-Nitrophenol, Pyrazole, Styrene, Tamoxifen, Theophylline 8-hydroxylation (at high conc.), Toluene.

Ethers

Diethylether, Methyl t-butyl ether, 1,1,2,3,3,3-hexafluoropropyl methyl ether

Fatty acids

Arachidonic acid w-1 and w-2 hydroxylation, Lauric acid w-1 hydroxylation

Halogenated and Nonhalogenated Alkanes and Alkenes

Acetoacetate, Acetol, Acetone, Acetonitrile (+catalase), Acrylonitrile, 1,3-Butadiene, Chloroform (to glutathione complex), Chloroform (low-affinity component), Chloromethane, Dibromoethane, 1,1-Dichloro-2,2,2-Trifluoroethane, 2,2-Dichloro-1,1,1-Trifluoroethane, Dichloromethane, 1,1-Dichloroethane, 1,1-Dichloroethylene, 1,2-Dichloropropane, N,N-Dimethylacetamide, N,N-Dimethylformamide, Enflurane, 1,2-Epoxy-3-butene, Ethane, Ethyl carbamate, Ethylene dichloride, Halothane, Hexane, β,β-Iminodipropionitrile, Methoxyflurane, Methyl formate, Methylenechloride, N-Methylformamide, Pentane, Seroflurane, 1,1,1,2,-Tetrafluoroethane, 1,1,2-Trichloroethane (TRI), 1,1,2,2-Tetrafluoro-1-1(2,2,2-trifluoroethoxy)ethane, Thioacetamide, Tirapazamine, 1,1,1-Trichloroethylene, Trichloroethylene, Vinyl chloride, Vinyl bromide

Nitrosamines, Azocompounds

Azoxymethane, N,N-Diethylnitrosamine, N,N-Dimethylnitrosamin, Methylazoxym ethanol, N-Nitroso-2,2-Dimethylmorpholine, N-Nitrosomethylbenzylamine, N-Nitrosopyrrolidine, N-Nitrosobis(2-Oxopropyl)Amine

Reducible Substrates

t-Butylhydroperoxide, Carbon Tetrachloride, Chromium [Cr(VI)], Cumyl Hydroperoxide, 13-Hydroperoxy-9,11-Octadecadienoic acid, 15-Hydroperoxy-5,8,11,13-Eicosatetraenoic acid, Oxygen.

Other groups have made attempts in the solubilisation of P450 enzymes by truncation of the putative N-terminal regions believed to be involved in anchoring the enzyme to the membrane. This has led to proteins still associated to the membranes, indicating that more interactions with the membrane are present. More recently, however, Jones and co-workers in USA (Shimoji et al., 1998) have successfully constructed and expressed a soluble and functional chimera of approximately 50% human P450 2C9 and 50% soluble bacterial P450cam. The enzyme was found to catalyse the oxidation of 4-chlorotoluene typical of human P450 2C9, using molecular oxygen and the reducing equivalents provided by the physiological electron transfer partners of the bacterial P450cam. Examples of engineered P450 enzymes which have been successfully expressed in Escherichia coli are shown in table 3

TABLE 3
Mammalian cytochrome P450s successfully expressed in Escherichia coli.

| CYP | Reference |
|---|---|
| 1A2 | Sandhu, et al 1994 |
| 1B1 | Schimada, et, al, 1998 |
| 2B1 | Szklarz and Halpert, 1997 |
| 2B2 | Strobel and Halpert, 1997 |
| 2B4 | Schumyantseva, et al, 1999 |
| 2B6 | Hanna, et al, 2000 |
| 2A4 | Sueyosh, et al, 1995 |
| 2A6 | Soucek, 1999 |
| 2C general | Richardson, et al, 1995 |
| 2C2 | Doray, et al, 1999 |
| CYP | Reference |
| 2C5 | Cosme and Johnson, |
| | 2000 |
| 2C10 | Sandhu, et al, 1993 |
| 2C11 | Licad-Coles, et al, |
| | 1997 |
| 2C17 | Sagara, et al, 1993 |
| 2D6 | Ellis, et al, 2000 |
| 3A4 | Gillam, et al, 1993 |
| 3A7 | Gillam, et al, 1997 |
| 4A5 | Hosny, et al, 1999 |
| P450c17 | Barnes, et al, 1991 |
| P450scc | Woods, et al, 1998 |

2E1 has been cloned and expressed by Umeno et al 1988.

A water soluble chimeric protein according to the invention comprises a scaffold domain and a monooxygenase haem containing domain, the scaffold domain is derived from BM3 and the haem domain is derived from a plant or an animal P450 enzyme.

In the invention the haem domain is derived from a plant or animal, usually mammalian, P450 enzyme, for instance by cleavage of the membrane-bonding portion of the physiological enzyme, which is hydrophobic and renders the P450 non-water soluble. Cleavage of this portion thus allows the P450 to be solubilised. Fusing the haem domain with a scaffolding domain from the self-sufficient bacterial redox protein BM3 provides further masking for the hydrophobic portions revealed by cleavage of the membrane binding component from the plant or animal P450, and provides a domain for interaction with other electron transfer components.

Preferably the chimeric protein comprises an integral electron transfer domain, that is a portion which is fused to the chimeric protein in a functional conformation. Generally this electron transfer portion comprises the physiological domain from a BM3 protein. Generally the electron transfer domain is a flavoprotein. Preferably the scaffold domain comprises at least 200, preferably at least 500 residues from a position at or adjacent to the N-terminal domain of wild type BM3.

The scaffold domain should not include the wild-type haem domain of BM3, i.e. up to residue number 471. Preferably the scaffold includes the portion from residue 472 to 652 comprising the FMN part of the haem reductase region as well as the portion from residue 652 to approximately the N terminal which comprises the FAD domain.

The haem containing domain of the plant or animal P450 generally comprises at least 200 contiguous residues from a wild type P450 enzyme, within which the haem-binding residues and the catalytic residues are located. The catalytic and haem-binding residues are generally found to be present at or near the C terminal of the wild type enzymes. Preferably the haem domain comprises at least 200 contiguous residues from a location at or adjacent to the C-terminal of a wild type P450 enzyme. Preferably the haem domain comprises more than 250, more preferably more than 300 contiguous residues. Preferably the haem domain is derived from one of the CYP's mentioned in Table 1 above. Since the CYP's of Table 3 have been cloned, the use of a domain derived from one of those CYPs is preferred. Most preferably the haem domain is derived from 2E1.

In some circumstances it may be desirable for the wild type enzyme to be mutated. For instance it may be desirable to introduce mutations at an active site, either for conferring suitable fusion properties with the scaffolding domain, or to analyse and investigate the effect of mutations on the activity of the enzyme and/or substrate binding properties. Since the main utility of the present invention is to investigate the binding site of physiological (wild type) enzymes, it is preferred that a low number of residues are mutated or deleted, and that any which are mutated are conservatively substituted. Preferably no more than twenty residues have been mutated relative to the filed type enzyme, more preferably fewer than ten, most preferably fewer than five residues have been deleted or altered.

In the invention, there is also provided a new process in which the chimeric protein is contacted with a substrate for the catalytic monooxygenase domain in a reaction mixture in the presence of oxygen whereby the substrate is oxidised to form an oxidised product. The process may be monitored by identifying the product of the reaction, or by monitoring the transfer of electrons from the haem containing domain to an electron transfer domain, or by monitoring oxygen consumption. The transfer of electrons may be monitored by including NAD(P)H in the reaction mixture and monitoring for production of NAD(P)⁺, for instance using the assay defined in our copending application number WO-A-0157236. Alternatively the electron transfer may be monitored by the use of an electrode capable of transferring electrons to or from the electron transfer domain of the chimeric protein, or an intermediate electron transfer component. This process is described and claimed in our copending PCT application filed on 3 Aug. 2002 based on earlier GB application number 0119042.0. The method is illustrated in the examples below, although for a wild type BM3 rather than the BM3-2E1 or other mammalian chimera.

The process of the invention may be used to monitor the presence or concentration of an analyte of interest which is the substrate. The process may alternatively be used to monitor metabolism of substrates of interest. The substrate may be any of those compounds mentioned in Table 2 above.

The invention is illustrated in the accompanying figures.

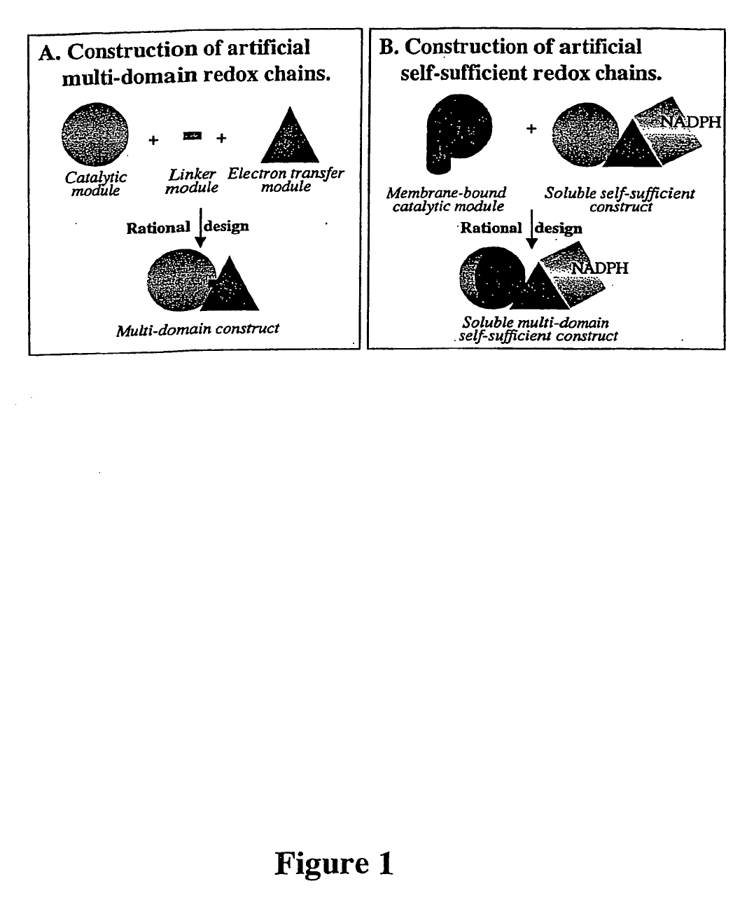

FIG. 1 shows the construction of P450 BM3 (A) to generate a P450 catalytic domain electrochemically accessible through the fusion with the electron transfer protein flavodoxin as claimed in our 3 Aug. 2002 application; (B) to solubilise the human membrane bound P450 2E1 by fusion with selected parts of the scaffold of the catalytically self sufficient P450 BM3 i.e. according to the present invention.

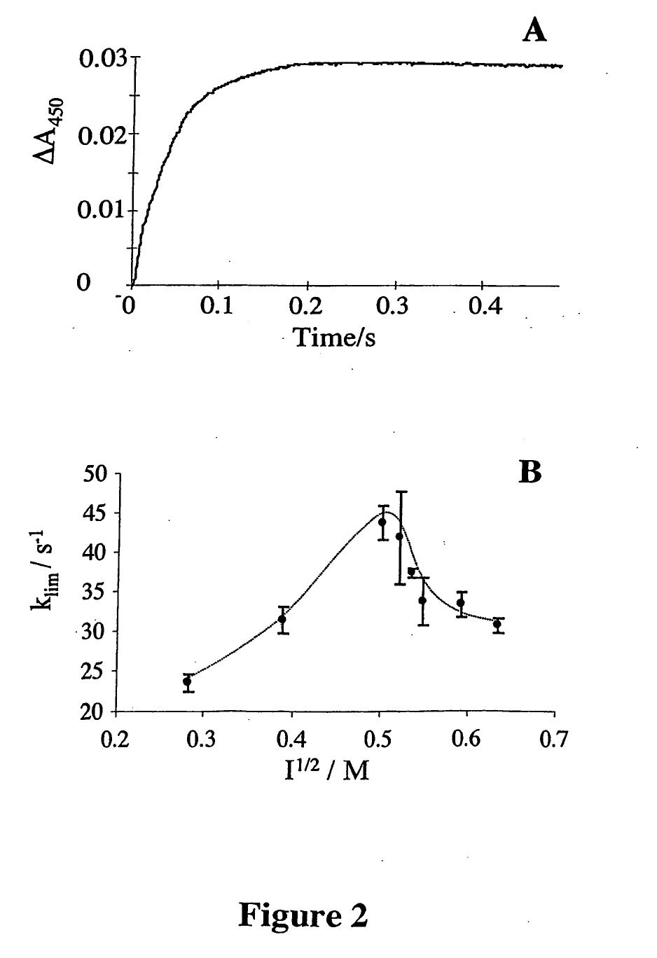

FIG. 2 shows (A) reduction of arachidonate-bound BMP (BMP-S) by flavodoxin semiquinone (FLD_{sq}) i.e. of the BM3-FLD chimera of FIG. 1A followed at 450 nm by stopped flow spectrophotometry in the presence of carbon monoxide. (B) Plot of the limiting pseudo-first-order rate constants (kₗᵢₘ) versus the square root of the ionic strength (I) for the reaction between FLD_{sq} and BMP-S.

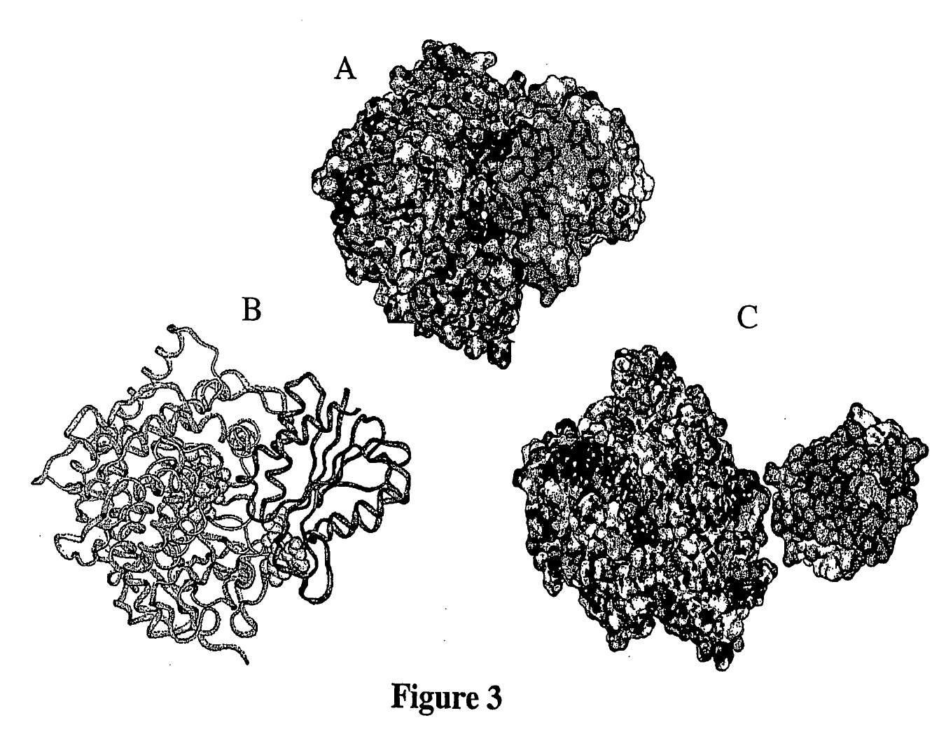

FIG. 3 shows 3D model of the complex between P450 BMP and FLD. (A) Side view of the docked complex. The van der Waals surface shows the electrostatic potentials calculated using DelPhi, where positive potentials are shown in darkest, negative potentials in mid-shading and neutral in white (contour scale±5 Kcal/mol). (B) Ribbon diagram of the complex in the same orientation as in A. The P450 BMP is shown in lighter shading to the left hand side, FLD is in darker shading to the right hand side, the FMN in space fill in the lower part of the FLD, and the haem in space fill in the centre of the BMP. (C) View of the open complex, with the same orientation as in A, but opened by a ±90° rotation to display the interface between the two proteins. These figures are reproduced in colour in Gilardi G. et al 2002.

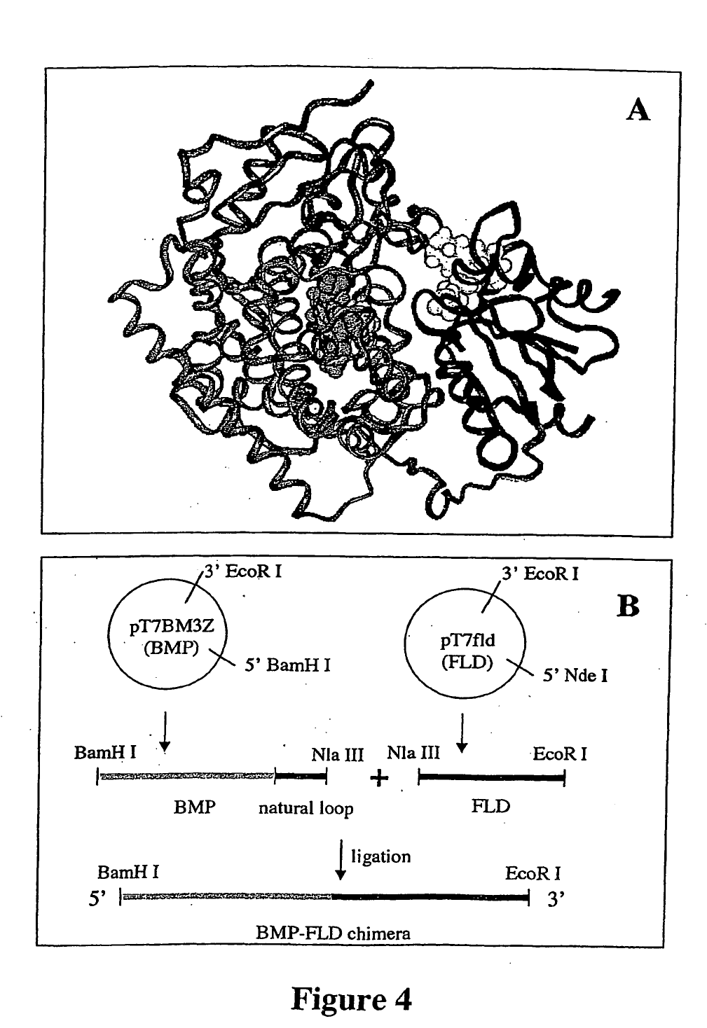

FIG. 4 shows (A) modelled structure of the BMP-FLD fusion protein with BMP domain in lighter shade ribbons, FLD domain in dark ribbon, haem in space filling centre of the BMP domain, cysteine 400 in lighter space filling adjacent the haem, FMN in lighter space filling at the top of the FLD and the connecting loop between the BMP and FLD (at the bottom of the model).

(B) Molecular biology approach to fuse the genes of BMP and FLD to generate the BMP-FLD chimera. The Nla III restriction sites were introduced by oligonucleotide directed mutagenesis. These figures are shown in Gilardi et al 2002 in colour.

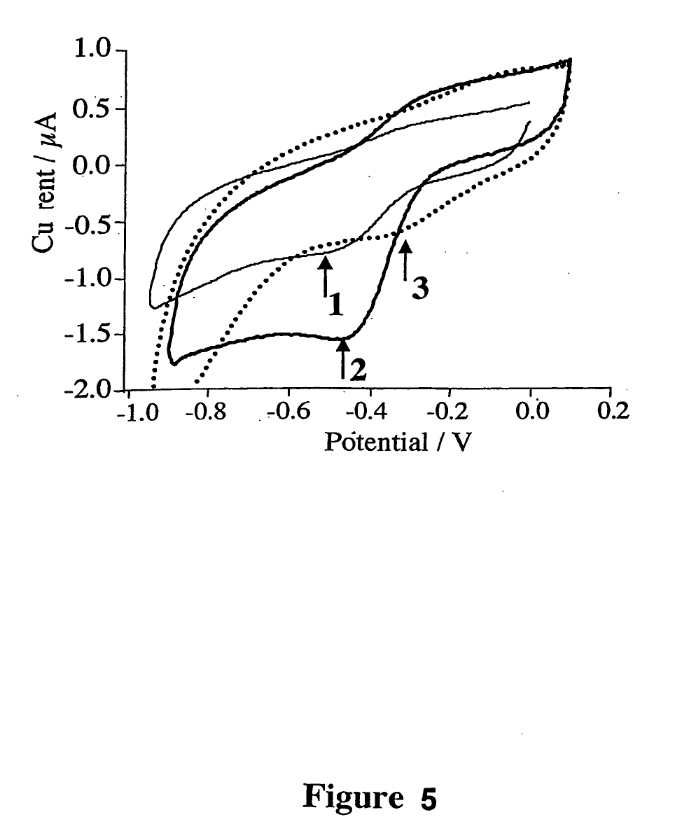

FIG. 5 shows cyclic voltammograms of BMP-FLD fusion protein in the absence (1, thin line) and presence (2, thick line) of neomycin on glassy carbon electrode. Addition of carbon monoxide. Shifts the peak to higher potentials (3, dotted line). Potentials are reported versus saturated calomel electrode.

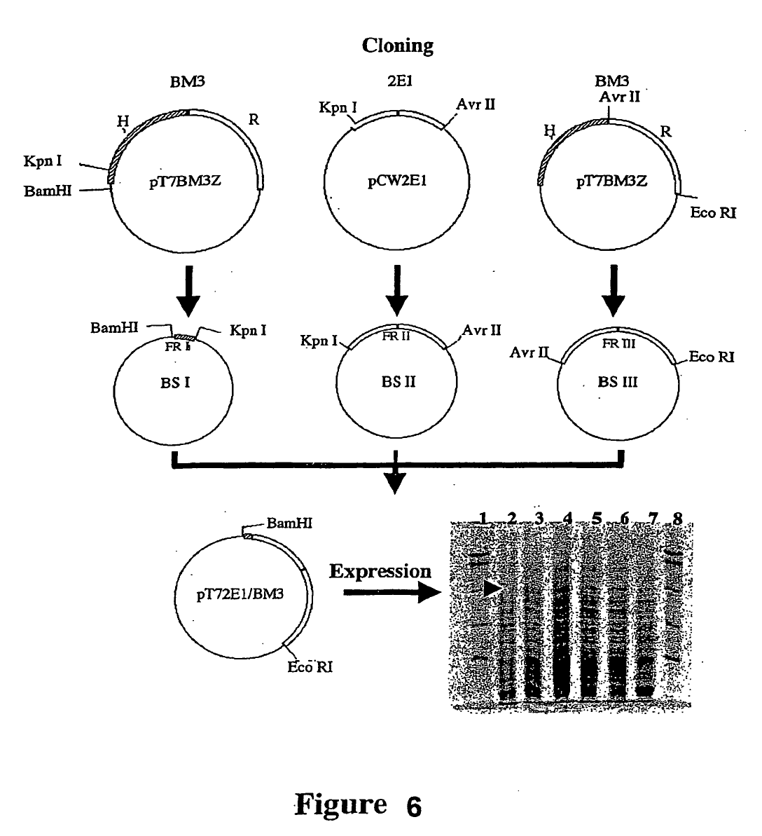

FIG. 6 shows at the top the cloning: strategy adopted to construct the first plasmid pT72E1/BM3 for the expression of the 2E1-BM3 chimera 2E1-BM3/1. Starting plamids are pT7BM3Z and pCW2E1 containing the genes of the P450 BM3 (H=haem domain, R=reductase domain) and human 2E1 respectively. Fragments I (FR I, Bam HI-Kpn I), II (FR II, Kpn I-Avr II) and III (FR III, Avr II-Eco RI) were cloned into the pBluescript SK(+/−) vector to give plasmids BSI, BS II and BS III respectively. Restriction sites were introduced by PCR using mutagenic oligonucleotides. The gene for the first 2E1-BM3 chimera was assembled by ligation of fragments I, II and III. At the bottom of the figure is shown expression, i.e. SDS-PAGE gel showing the expression of the 2E1-BM3 chimera. The arrow indicates the position of the 2E1-BM3 chimera (118 kDa). Lanes 1 and 8: molecular weight markers (from bottom): 53, 76, 116, 170, 212 kDa. Lane 2: cell lysate of BL21 (DE3) Cl cells. Lane and 4: cell lysate of BL21 (DE3) Cl cells transformed with the pT72E1/BM3 plasmid that have been induced with 1 mM IPTG (cell growth for 20 h at 28C). Lane 5: same as lane 3 and 4, but after 10,000 g centrifugation to remove membrane fractions and inclusion bodies). Lane 6: same as lane 3 and 4, but after a 100,000 g centrifugation. Lane 7: pellet after the 100,000 g centrifugation.

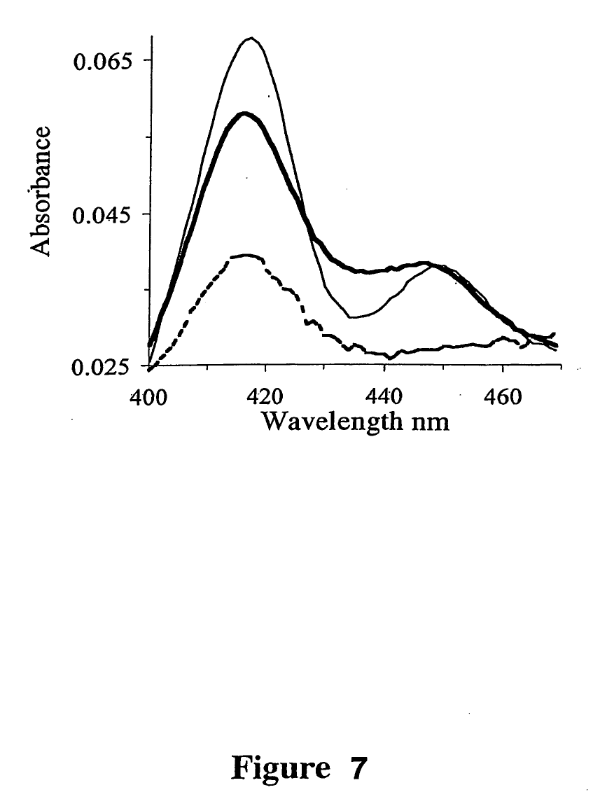

FIG. 7 shows absorption spectra of cleared lysates of E.coli cells non-transformed (dotted line), transformed with the BMP-FLD plasmid (thin line) and the first 2E1-BM3 plasmid (2E1-BM3/1) (thick line) after reduction with sodium dithionite and bubbling with carbon monoxide.

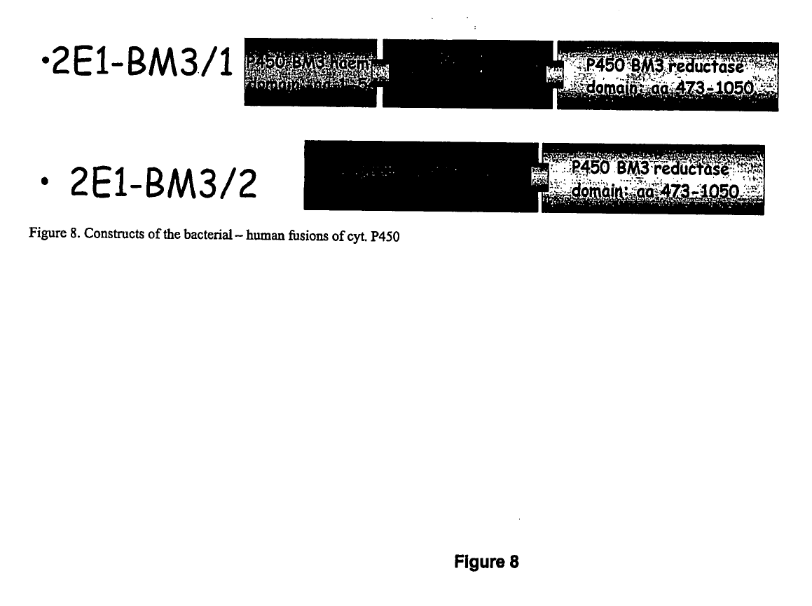

FIG. 8 shows diagrammatic representations of the constructs of the two 2E1-BM3 chimeras (2E1-BM3/1 and 2E1-BM3/2) made in the examples.

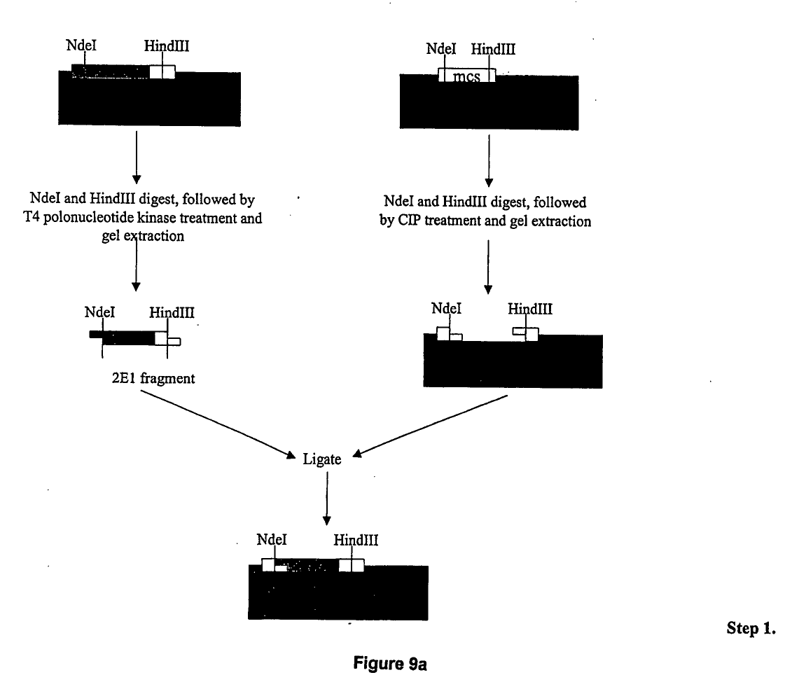

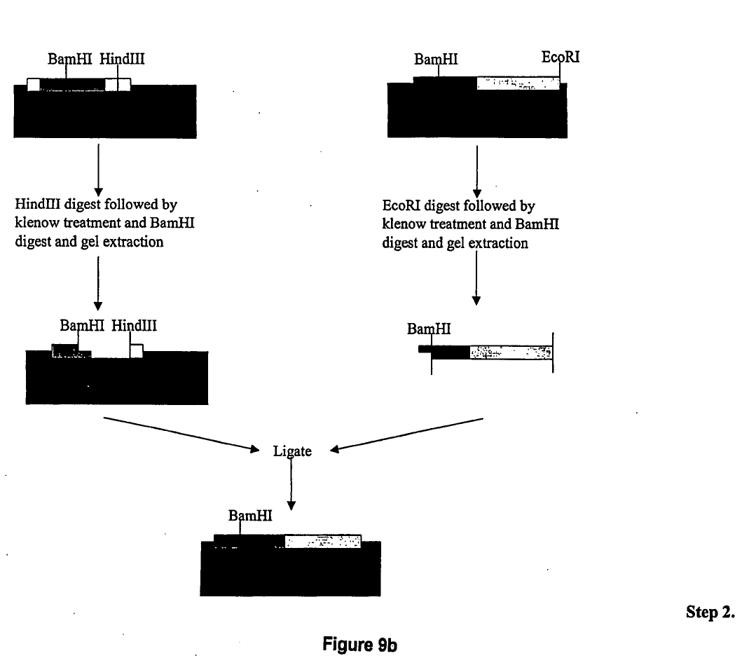

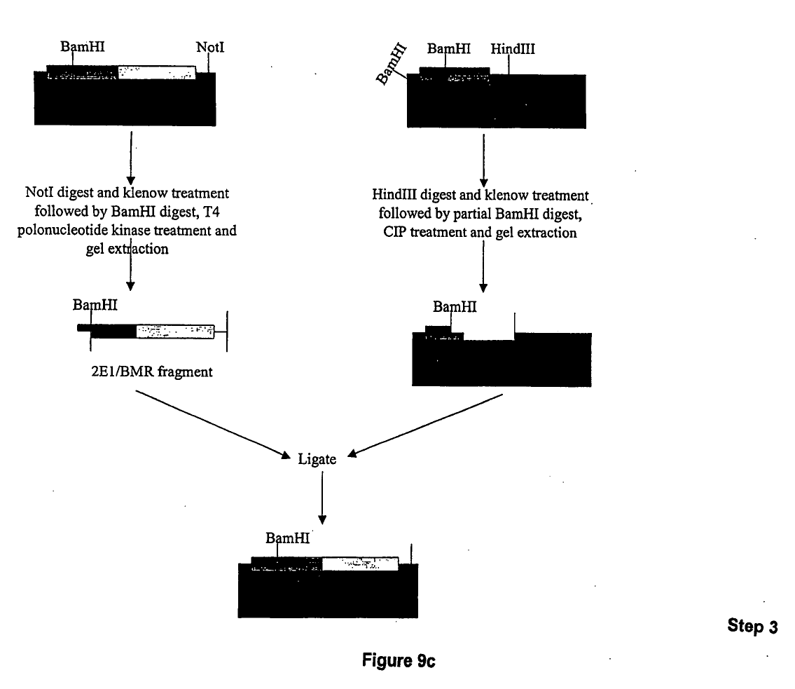

FIGS. 9a-c show the cloning steps used in the generation of the second 2E1-BM3 chimera, i.e. that illustrated in FIG. 8.

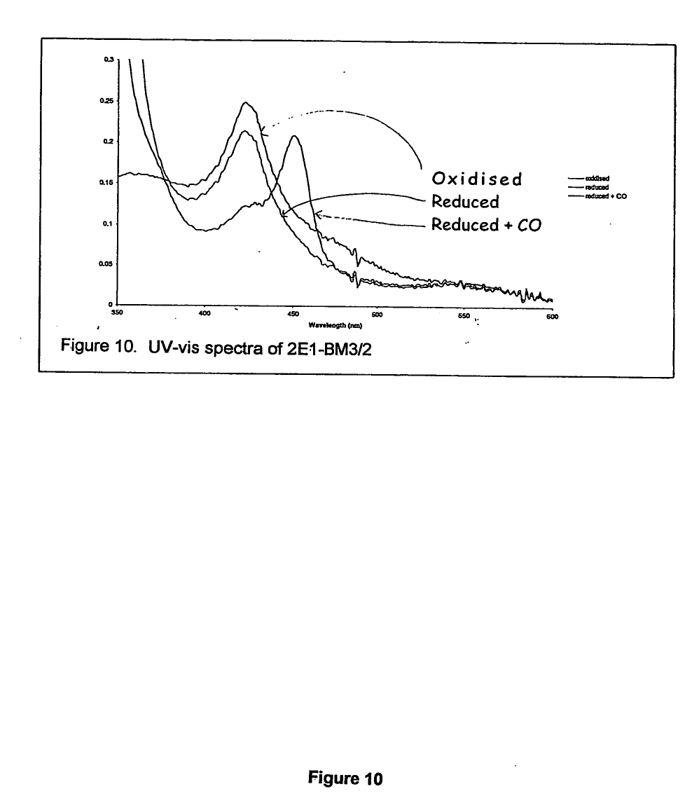

FIG. 10 shows that uv-visible spectrum of the second 2E1-BM3 chimera (2E1-BM3/2) with the haem group in oxidised form, reduced form and reduced form in the presence of carbon monoxide.

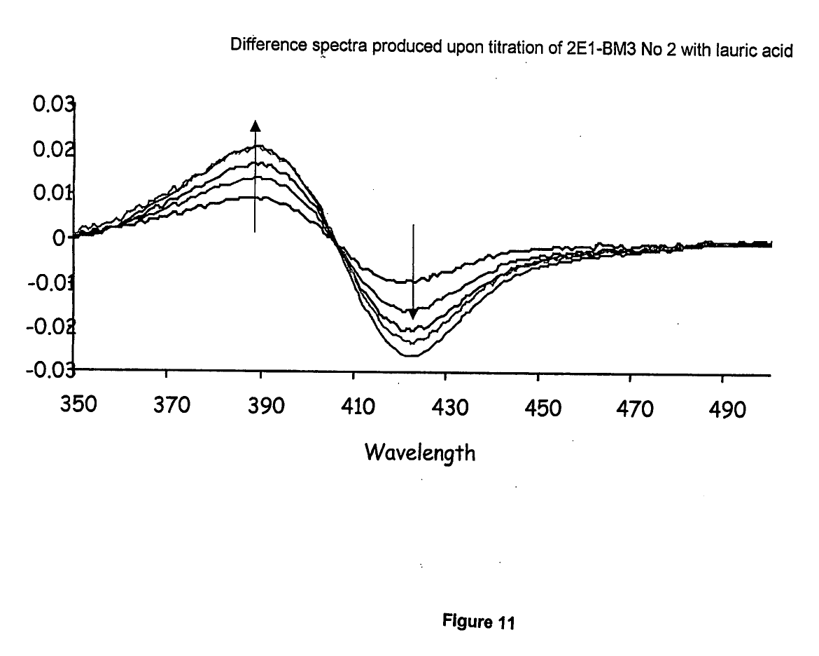

FIG. 11 shows the uv-visible difference spectrum for the second 2E1-BM3 chimera (2E1-BM3/2) in the presence of lauric acid, showing the increase of absorbance at 390 nm (high spin haem iron) and the decrease at 420 nm (low spin haem iron) upon increasing the concentration of lauric acid.

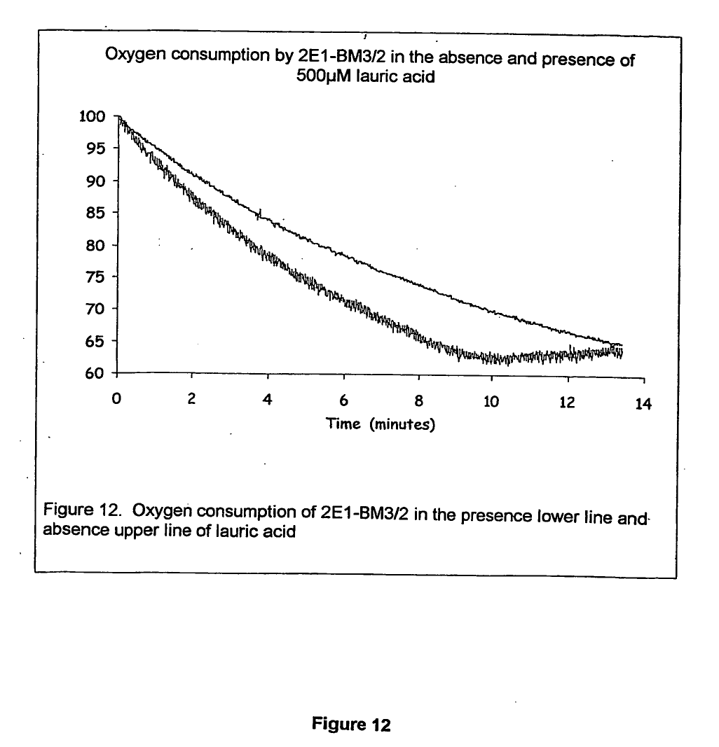

FIG. 12 shows the oxygen consumption of the second 2E1-BM3 chimera in the presence of lauric acid.

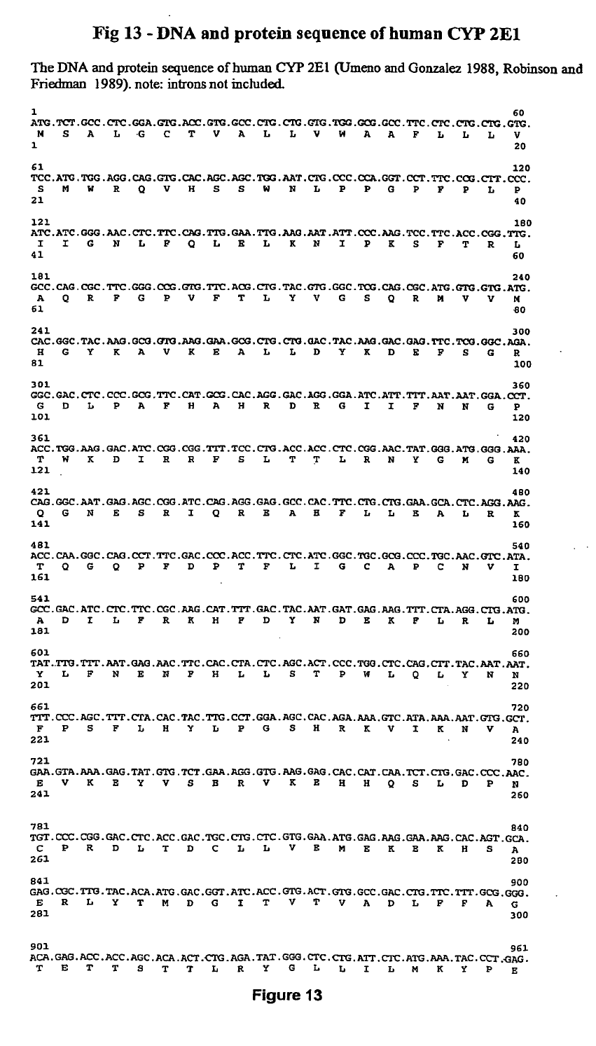

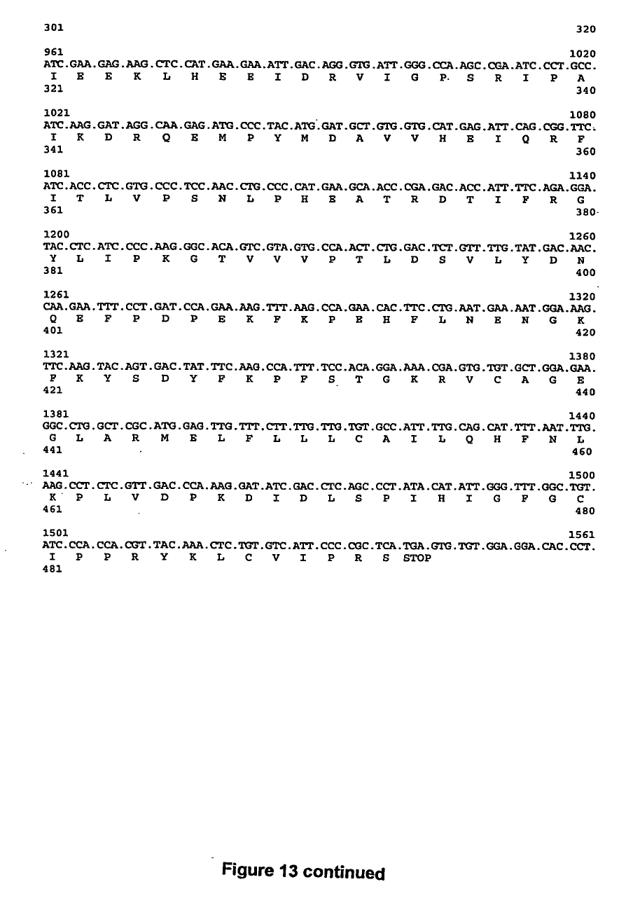

FIG. 13 shows the sequence of human CYP 2E1 from Umeno et al 1988 with introns not included.

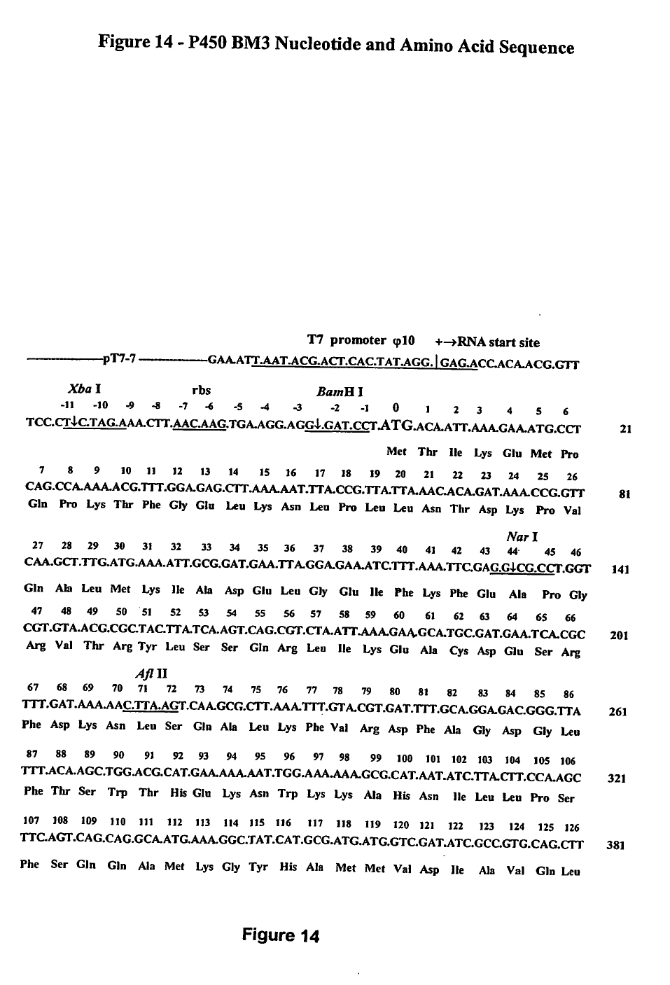

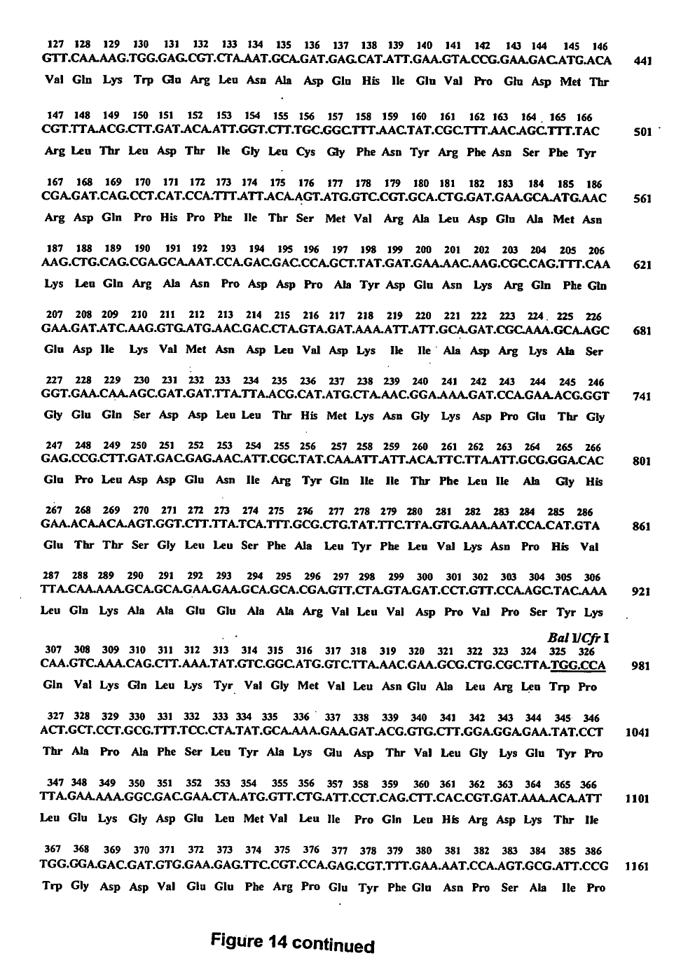

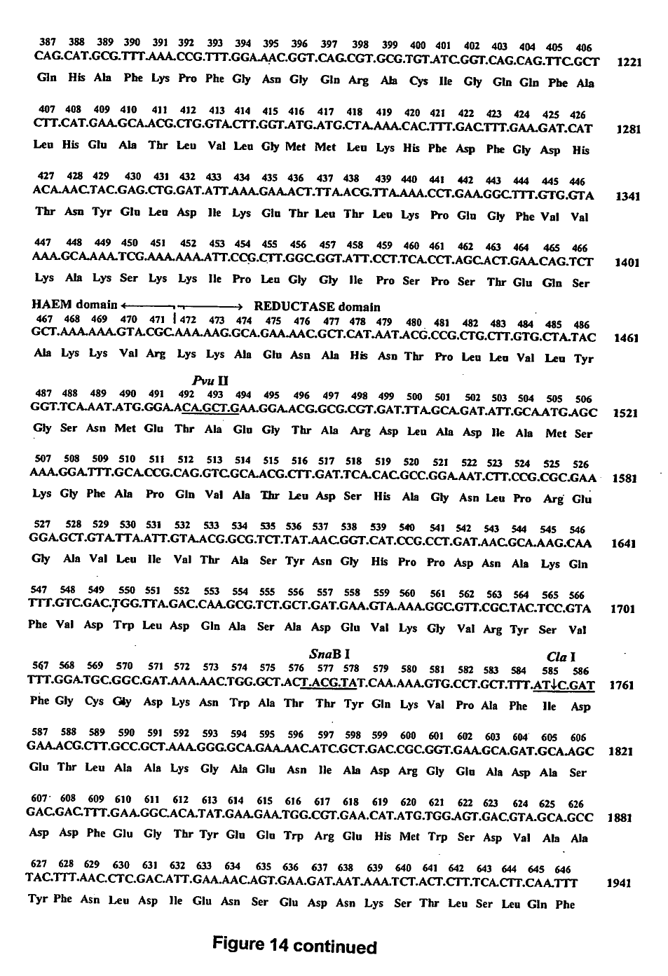

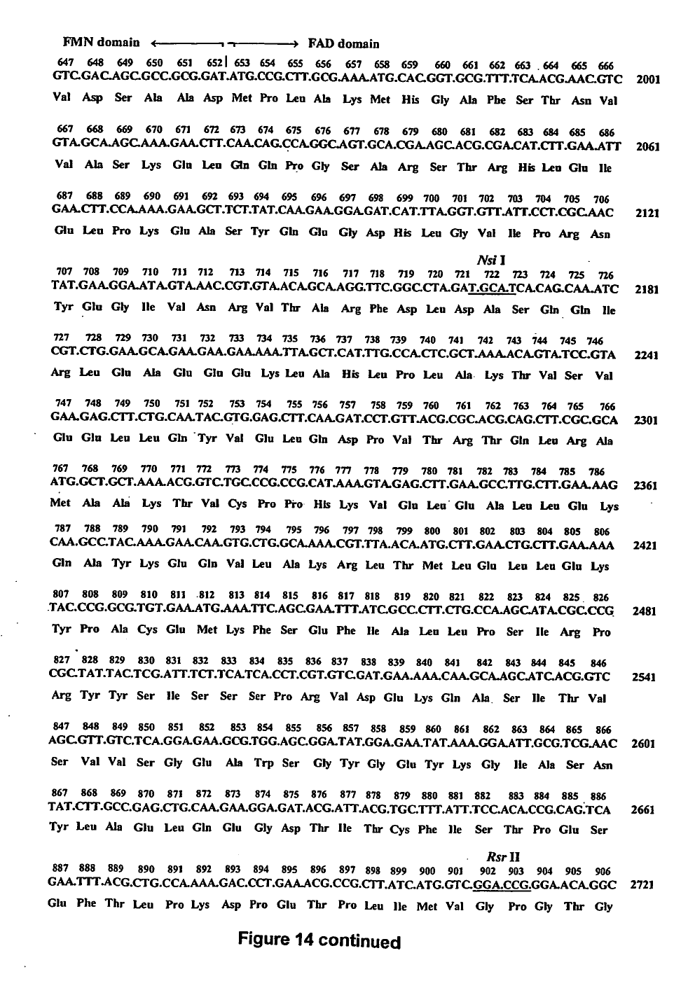

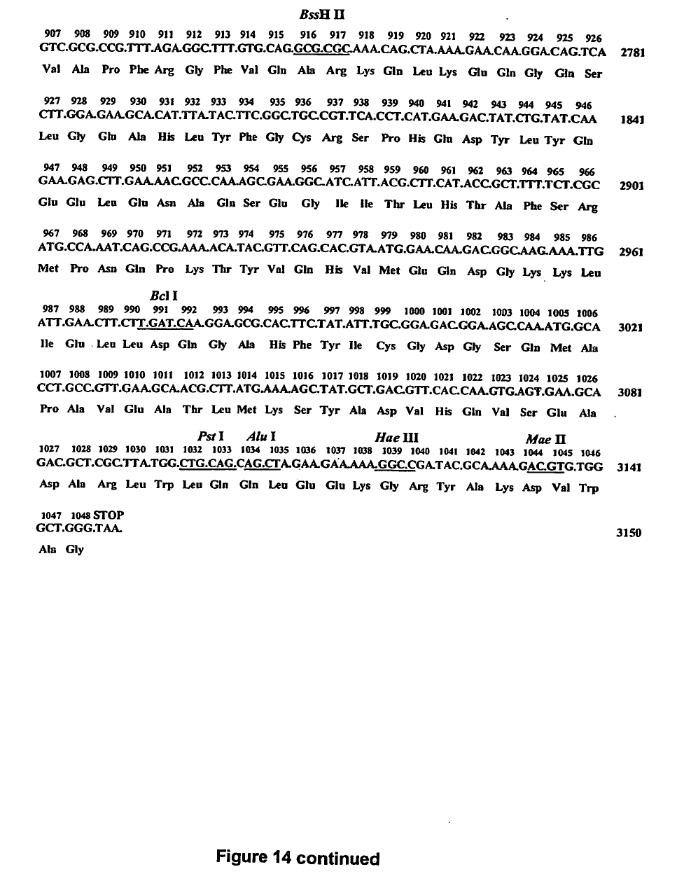

FIG. 14 shows the sequence of B. megaterium P450 BM3. The 5-kb DNA fragment containing the gene encoding P450 BM3 was isolated and sequenced by Fulco and co-workers. (Ruettinger et al., 1989). The nucleotide sequence was submitted to the GenBank™/EMBL Data Bank with accession number J04832. The P450 BM3 coding region plus some regulatory regions 5′ to the P450 BM3 gene on the pT7Bm3Z construct (Darwish et al., 1991) are given in the figure.

The open reading frame consisting of 3,147 bp is given. 5′ regulatory regions of the construct pT7Bm3Z starting from the T7 RNA φ10 promoter are also displayed. The number above each base triplet is the serial number of the amino acid counting the initial Met as zero (Thr 1 was the first residue detected in the NH₂-terminus of P450 BM3 by protein sequencing (Ruettinger et al., 1989)). To the right of the sequence is given the serial number of the base (starting from the start codon, every 60 bases). Restriction sites unique within the gene are underscored and indicated over the corresponding bases. The conventional division between the haem and the reductase domain between Arg471 and Lys472, as well as the division between the FMN and the FAD domain between Asp652 and Met653 are also indicated.

The invention is illustrated further in the accompanying examples.

Materials & Methods

Electron Transfer Measurements Between P450BM3 Haem Domain (BMP) and Flavodoxin (FLD).

All absorbance measurements were carried out using a Hewlett-Packard 8452 diode array spectrophotometer. The wild type flavodoxin from D. vulgads (FLD, 4.9 μM) in 5 mM potassium phosphate buffer pH 7.3 was photoreduced in the presence of 2.5 μM deazariboflavin (dRf) and 0.85 mM EDTA (sacrificial electron donor) to its semiquinone form (FLD_{sq}, equations [1] and [2] of the results section). Kinetic measurements were carried out following the reduction of the arachidonate bound BMP under carbon monoxide atmosphere, monitoring the absorbance at 450 nm in a Hi-Tech SF-61 stopped flow apparatus with a 1 cm path length cell, at 23° C. The typical arachidonate bound BMP concentration was 1 μM, and that of FLD was varied between 2-20 μM (equation [3] of the results section). Special care was taken to achieve anaerobic conditions by bubbling all solutions with argon. The method is based on that of Heering-Hagen 1996.

Construction and Expression of the BMP-FLD Chimera.

The BMP-FLD fusion complex was constructed by introducing a Nla III site both at the 3′ end of the loop of P450 BM3 reductase gene in pT7BM3Z (Li et al., 1991) and 5′ end of the pT7FLD gene (Krey et al., 1988, Valetti et al., 1998). This was carried out by PCR using the mutagenic oligonucleotides sequence ID's Nos 1 and 2 listed in Table 4. The two genes were digested with Nla III endonuclease followed by a ligation step. The expression and purification of the wild type (wt) P450 BM3 and of the BMP-FLD chimera were carried out according to published protocols (Li et al., 1991, and Sadeghi et al., 2000a, respectively).

Electron Transfer Measurements on the BMP-FLD Fusion Protein.

Steady-state photo-reduction of 4 μM BMP-FLD fusion protein was performed in 100 mM phosphate buffer pH 7 containing 5 μM deazariboflavin and 5 μM EDTA, under strict anaerobic conditions; photo-irradiation was carried out using a 100 W lamp. Laser flash photolysis was carried out as previously described (Hazzard et al. 1997). The BMP-FLD fusion protein (5 μM) was kept under strict anaerobic conditions in carbon monoxide saturated 100 mM phosphate buffer pH 7, containing 100 μM of deazariboflavin and 1 mM EDTA.

Electrochemical Experiments on the BMP-FLD Fusion Protein.

All electrochemical experiments were carried out with the Autolab PSTAT10 controlled by the GPES software (Eco Chemie, Utrecht, NL). The staircase cyclic voltammetry was performed in a Hagen cell (Heering and Hagen, 1996) where the working electrode was glassy carbon disc with a platinum wire as the counter. The working electrode was activated and polished as previously described (Heering and Hagen, 1996). The reference electrode was Saturated Calomel with a potential of +246 mV versus the normal hydrogen electrode (NHE). All measurements were performed under strict anaerobic conditions with protein concentrations of 30 μM in 50 mM HEPES buffer pH 8.0, at 7° C.

UV-visible Spectra of BM3 Chimeras 5.4 nmol of P450 BM3 in 50 mM HEPES buffer, pH 8.0 was reduced by the addition of 1 μl of a saturated solution of sodium dithionite. Gentle bubbling with carbon monoxide followed for about 1 min.

Molecular Modeling.

All modelling studies and calculations were performed using the Biosym/MSI software installed on an SGI Indigo2 workstation, IRIX 6.2. Surface electrostatic potentials were calculated using the DelPhi 2.0 module under Insight II environment. DelPhi calculations were performed using a dielectric constant of 2.0 for the solute and 80 for the solvent with an ionic strength of 100 mM; solvent radius was set at 1.4 Å and ionic radius at 2.0 Å. The Poisson-Boltzmann algorithm was applied in its non-linear form with a limit of 2000 iteration and convergence of 0.00001 to a grid of resolution ≦1.0 Å, centred around the protein. The minimal distance between the molecular surface and the grid boundary was 15.0 Å. Only formal charges were taken into account: the C- and N-terminus and the Glu, Asp, Arg and Lys side-chains were considered to be fully ionised, with the FMN phosphate and the haem iron (Fell) also included in the calculation. The solvent exposure was calculated using the Connolly algorithm (Connolly, 1983), with a probe of 1.4 Å radius . The Protein Data Bank (pdb) files used were the oxidised form of FLD (Watt et al., 1991), the P450terp (Hasemann et al., 1994), P450cam (Poulos et al., 1986), P450eryF (Cuppvickery and Poulos, 1995) and the haem domain of P450 BM3 (Ravichandran et al., 1993; Li and Poulos, 1997; Sevrioukova et al.,1999). The model of the human P450 2E1 was built by using the application Homology within the program Insight. II 95.0 (Byosim/MSI), and the preliminary model was finally refined and energy minimised by submitting it to the module Discover of Insight II.

Construction and Expression of 2E1-BM3 Chimera No. 1.

The DNA fragments used for the construction of the 2E1-BM3 chimera No. 1 were obtained from plasmids pT7BM3Z for P450 BM3 (Darwish et al., 1991) and pCW2E1 for P450 2E1 (Gillam et al., 1994). Suitable restriction sites were inserted by site-directed mutagenesis using the PCR enzyme Vent DNA polymerase (New England Biolabs) with mutagenic oligonucleotide primers. Sequence ID's 3 to 8 listed in Table 4. The amplified PCR fragments with the suitable restriction sites were cloned into the pBluescript SK (+/−) amplification vector (Stratagene) following the procedure shown in FIG. 6. The pT72E1/BM3 plasmid was expressed under the control of the T7 promoter for inducible expression in Escherichia coli BL21 (DE3) Cl (Stratagene). 1 ml of an overnight culture of LB-ampicillin (100 μg/ml) was used to inoculate 100 ml of LB-ampicillin. This was grown at 37° C. until the optical density at 600 nm (OD₆₀₀) was 1. This culture was then used to inoculate 9 l of LB-amp and IPTG (1 mM) and further ampicillin were added at OD₆₀₀ of 0.4-0.6; cell growth was then continued at 28° C. for 21 h. Cells were harvested by centrifugation at 5000 rpm for 15 min at 4° C., the cell pellet was resuspended in 100 mM potassium phosphate pH 7.0 (buffer A) and repelleted. The cells were resuspended in buffer A using 1 ml of buffer per gram of cells, lysed by sonication and centrifuged at 10,000 rpm for 20 min. The cleared cell lysate was ultra-centrifuged at 38,000 rpm for 1 h to separate the membrane fraction from the cytosol (soluble fraction). The soluble fraction was then loaded onto a DEAE sepharose fast flow column (Pharmacia) pre-equilibrated with buffer A. The 2E1-BM3 chimera was eluted with a 100-500 mM gradient of potassium phosphate pH 7.0.

Construction and Expression of 2E1-BM3 Chimera No. 2

2E1-BMR was engineered simply by restriction digest of the existing clones, i.e. no mutagenic PCR (hence primers) were required. The cloning steps followed are illustrated in FIGS. 9a-c. The steps for the construction are as follows:

Step 1: pET30b2E1

We started from the pCW2E1 (red in fig) from which the wild type 2E1 was transferred to the commercially available pET30b vector. This is missing the first 21 aminoacids (called N-terminal modification).

Step 2: pET2E1-BM3/2

The pET30b2E1 construct was used as a template on which to insert the Bam HI-Eco RI fragment from pT72E1BM3. Essentially this fragment replaced part of the 2E1 gene in pET30b2E1 with the addition of the BMR contained in pT72E1BM3. This gave the construct pET2E1/BM3/2. In theory this should have been ready for expression. In practice it gave inclusion bodies.

Step 3: pCW2E1-BM3/2

To improve expression, the 2E1-BM3/2 construct was subcloned into the pCW vector starting from the original pCW2E1. This was achieved with a BamHI cut combined with a blunt end ligation at the C-terminus. The clone was expressed as for the first 2E1-BM3 chimera.

Oxygen Consumption of 2E1-BM3/2 Chimera in Presence of Substrate

940 ml of 100 mM potassium phosphate buffer pH 8 was added to the oxygen electrode chamber (Oxygraph system by Hansatech Instr. Ltd.) and stirred for 10 minutes at 25° C. (with or without 500 mM lauric acid dissolved in 50 mM potassium carbonate). 50 ml of protein in the same phosphate buffer was then added using a Hamilton syringe (0.4 mM final protein concentration) and the mixture stirred for 3 minutes. 5 ml of NADPH was then added (75 mM final concentration) and oxygen concentration measured till consumption had stopped. A further 5 ml of NADPH was then added (75 mM final concentration) and oxygen concentration measured till consumption had stopped.

Results

Assembling Artificial Redox Chains

The suitability of flavodoxin from D. vulgaris (FLD) and the haem domain of cytochrome P450 BM3 from B. megaterium (BMP) as electron transfer and catalytic modules to be used for the covalent assembly of a multi-domain construct was tested. The electron transfer (ET) between the separate proteins was studied by stopped-flow spectrophotometry. Flavodoxin (FLD_{q}) was reduced anaerobically under steady state conditions to its semiquinone form (FLD_{sq}) in one syringe of the stopped-flow apparatus by the semiquinone radical of deazariboflavin (dRfH) produced by photo-irradiation in the presence of EDTA. The reaction scheme studied is summarised in the following equations (Sadeghi et al, 1999):

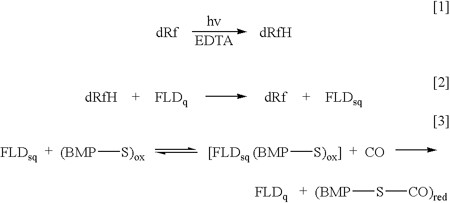

Under pseudo-first order and saturating conditions, the ET process of the FLD_{sq}/(BMP-S)ₒₓ redox pairs showed an increased of the absorbance at 450 nm (FIG. 2A). This is consistent with the reduction of (BMP-S)ₒₓ, that promptly forms the carbon monoxide adduct responsible for the absorbance at 450 nm. The pseudo-first order rate constant (k_{obs}) was calculated by fitting the data points to a single exponential component. When the concentration of FLD_{sq} was varied between 2-20 μM, the k_{obs} was found to follow a saturating behaviour consistent with the formation of a complex between the two proteins. Fitting the data points of the k_{obs} versus the concentrations of FLD_{sq} to a hyperbolic function led to the limiting rate constant, kₗᵢₘ, of 43.77±2.18 s⁻¹ and to the apparent dissociation constant, Kₐₚₚ, of 1.23±0.32 μM at an ionic strength of 250 mM in 10 mM phosphate buffer, pH 7.3.

An important factor for achieving efficient ET is the formation of an ET competent complex between the redox pairs. The effect of the electrostatic forces in producing the complexes between BMP and FLD was studied by changing the ionic strength of the protein solutions. The resulting kₗᵢₘ values plotted against the square root of the ionic strength, I, showed the bell-shaped trend shown in FIG. 2B. This is usually due to hydrophobic as well as electrostatic interactions taking part in the formation of the complex (Sadeghi et al., 2000b). This was confirmed by the calculation of the surface potentials of the two proteins shown in FIG. 3.

The availability of the 3D structures of chosen protein modules allows the use of computational methods for generating a 3D model of the possible complexes. The structure of such models is important in this work for the rational design of the covalently linked assembly described here.

A model for the FLD/BMP complex (FIG. 3B) was generated by super-imposition of the 3D structure of FLD on that of the truncated P450 BM3 (Sevrioukova et al., 1999). The distance between the redox centres in this complex is 18 Å, which is comparable with that found in the structure of the truncated P450 BM3 (Sevrioukova et al., 1999). However, an alternative model is also possible, where the FMN region of FLD is docked in the positively charged depression on the proximal BMP surface, around the haem ligand cysteine 400. This model brings the two cofactors at a closer distance of <12 Å. The two possible models may reflect the presence of dynamic events accompanying the formation and reorganisation of the ET competent complex that has also been postulated for the natural P450-reductase complex (Williams et al., 2000).

The model of the ET competent complex described above was used to generate a covalently linked complex of BMP-FLD. This was achieved by linking a flexible connecting loop introduced by gene fusion as shown in FIG. 4B. This method offers the advantage of keeping the two redox domains in a dynamic form. The fusion of the BMP-FLD system was carried out at DNA level by linking the BMP gene (residues 1-470) with that of FLD (residues 1-148) through the natural loop of the reductase domain of P450 BM3 (residues 471-479). The 3D model of this fusion protein is shown in FIG. 4A. The gene fusion was achieved by ligation of the relevant DNA sequences with engineered Nla III restriction sites, as shown in FIG. 4B.

The fusion gene was heterologously expressed in a single polypeptide chain in E.coli BL21 (DE3) Cl. The absorption spectra of the purified chimeric protein indicated the incorporation of 1:1 haem and FMN. Moreover, the reduced protein was able not only to form the carbon monoxide adduct with the characteristic absorbance at 450 nm, but also to bind substrate (arachidonate) displaying the expected low- to high-spin transition from 419 nm to 397 nm, indicating that this covalent complex is indeed a functional P450. The integrity of the secondary structure of the BMP-FLD fusion protein was confirmed by CD spectroscopy (data not shown), with a ˜2% increase in the a-helix content when compared to the BMP, probably due to the addition of the engineered loop. The spectroscopic data show that the fusion protein is indeed expressed as a soluble, folded and functional protein (Sadeghi et al., 2000a).

The presence of intra-molecular ET in the BMP-FLD fusion protein, from the domain containing the FMN to the domain containing the haem, in the presence of substrate, was studied under steady-state conditions. The flavin domain was photo-reduced by deazariboflavin in the presence of EDTA under anaerobic conditions. The subsequent ET from the flavin domain to the haem was followed by the shift of the haem absorbance from 397 nm to 450 nm in carbon monoxide saturated atmosphere. The kinetics of the intra-molecular ET within the BMP-FLD fusion protein was studied by transient absorption spectroscopy. In the experimental set up, the FMN-to-haem ET was followed by the decrease in absorbance at 580 nm of the FLD_{sq}. The ET rate measured was found to be 370 s⁻¹. This-value is comparable to that measured for the intra-protein ET from FMN to haem domain of truncated P450 BM3 (250 s⁻¹) in which the FAD domain was removed (Hazzard et al., 1997). These results are extremely encouraging because they demonstrate the functionality of the BMP-FLD fusion protein to be equivalent to the physiological protein.

Preliminary electrochemical experiments of the BMP-FLD fusion protein were carried out using a glassy carbon electrode. The cyclic voltammograms (cv) of both the BMP-FLD fusion protein and BMP are shown in FIG. 5. While no current was observed for P450 BM3 enzyme on the bare glassy carbon electrode, the BMP-FLD shows measurable redox activities (thin line, FIG. 5). In particular, the BMP-FLD fusion protein interacts better with the electrode as measured by the larger current (thick line, FIG. 5) observed in the presence of neomycin, a positively charged aminoglycoside which is believed to overcome the electrostatic repulsion between the negatively charged FLD and the negatively charged electrode surface (Heering and Hagen, 1996). The enhancement of the current obtained in the presence of neomycin observed for BMP-FLD supports the hypothesis that FLD assists the electrochemical contact between the electrode and BMP. Efforts are currently made to achieve full electrochemical reversibility, as the lower current observed in the oxidative scan is consistent with oxygen leakage in the electrochemical cell; The results are consistent with the electrochemical response of the P450 haem, as supported by the shift at higher potentials in the cv obtained after addition of carbon monoxide (dotted line, FIG. 5).

The data presented in this section prove that indeed non-physiological electron transfer between the BMP catalytic module and the FLD electron transfer module is possible, and the covalently linked multi-domain construct BMP-FLD exhibits improved electrochemical properties.

Assembling Human/bacterial 2E1-BM3 P450 Enzymes

2E1-BM3/1

This section reports on the design, construction and expression of a human/bacterial chimeric P450 between the P450 BM3 from Bacillus megaterium and the human P450 2E1 (2E1-BM3), to obtain a soluble construct. This was obtained by fusing part of the human P450 2E1 with portions of P450 BM3 guided by rational design on the 3D structure/model of the two proteins, as shown in the scheme of FIG. 1B. Whilst the 3D structure of the haem domain and truncated form of the soluble cytochrome P450-BM3 is known from X-ray crystallography (Ravichandran et al. 1993; Li and Poulos, 1997; Sevrioukova et al.,1999), the human 2E1 enzyme is membrane bound and no structural information is available. For this reason, a three-dimensional model of P450 2E1 was built in order to assist with the rational design of a chimera that retains the catalytic elements of the 2E1 and eliminates the membrane-associated N-terminal parts of 2E1 by replacing them with the soluble parts of the BM3. The procedure followed in the design of the 3D model is as follows. (1) Four related proteins of known X-ray structure were chosen (P450terp, P450cam, P450eryF and the BMP domain of P450 BM3) and their sequences were aligned with that of P450 2E1, using the structurally conserved regions (SCRs). (2) The coordinates of the structurally variable regions (VRs) were assigned using different templates for different VRs, depending on the degree of homology (when the VRs were of different length the coordinates were assigned by a loop search). (3) A possible initial conformation of the side chains was searched and the coordinates of the more flexible N- and C-terminal regions were arbitrarily assigned. (4) Incorrect steric contacts (bumps) were corrected by manually orienting the involved rotamers. (5) The model was refined and energy minimised.

Analysis of the quality of the 3D model was carried out by using Biotech Validation Suite for Protein Structures (Laskowski et al., 1993; http://biotech.ebi.ac.uk:8400) with a resolution of 2.5 Å. The statistics of the Ramachandran plot showed that 92.4% of the residues are in the most favoured regions of the plot. Also, the overall G factor, an indicator of quality of the stereochemical properties (torsion angles and covalent geometry) of the model, was found to be −0.51. These parameters indicate that the current 2E1 model is suitable for the objectives set in this work, namely the identification of the residues of the human 2E1 enzyme to be fused with the P450 BM3 to achieve solubility. Further refinement of the model is currently being carried out, as this will allow future detailed structural/functional studies related to substrate binding and catalytic properties.

The information gained from the model of the wild type P450 2E1, together with previous works on isozymes (Shimoji et al., 1998; Pemecky, 1995, Nelson and Strobel, 1998; Jenkins and Waterman, 1998), was used to design the chimeric cytochrome P450, the 2E1-BM3. On this basis, the first 54 residues at the N-terminal of P450 BM3 (fragment I), the sequence of P450 2E1 from residue 81 to the C-terminal (fragment II) and the whole reductase domain of P450 BM3 (fragment III) were chosen to be fused at DNA level to generate the soluble 2E1-BM3 chimera. To this end, the parental P450-BM3 and 2E1 genes were amplified from plasmids pT7BM3Z and pCW2E1 by using the suitable oligonucleotide primers of Table I with the procedure illustrated in FIG. 6.

Fragment I was isolated from the pT7BM3 plasmid containing the whole sequence of the P450 BM3 gene. BamHI and KpnI restriction sites were respectively inserted at its ends. Fragment II was isolated from the pCW2E1 vector containing the human P450 2E1 gene sequence and KpnI and AvrII restriction sites were inserted at its ends. The fragment digested with KpnI was cloned into pBluescript previously digested with KpnI/EcoRV. Fragment III was isolated from the same pT7BM3 vector used for fragment I. AvrII and EcoRI sites were inserted at its ends and the fragment digested with EcoRI was cloned into pBluescript previously digested with EcoRV and EcoRI enzymes.

After amplification by PCR the three fragments were isolated from their respective pBluescript vectors using the designed restriction sites (respectively BamHI/KpnI for fragment I, KpnI/AvrII for fragment II and AvrII/EcoRI for fragment III). Fragment II and Fragment III were ligated in sequence into pBluescript in an intermediate step. The whole construct of 1350 base pairs, containing the three fragments together, was finally ligated into the pT7 vector to give the pT72E1/BM3 plasmid for the inducible expression in E.coli.

The 2E1-BM3 chimera 2E1-BM3/1 was successfully expressed in a soluble form by using E.coli BL21 (DE3) Cl cells. Results from the expression experiments are shown in FIG. 6. Expression of the 118 kDa 2E1-BM3, indicated by an arrow in FIG. 6, is shown in lane 3. The presence of the 2E1-BM3 chimera in the soluble fractions after ultra-centrifugation of the cell lysate (lane 5 and 6) and its absence from the insoluble membrane fractions (lane 7) shows that the protein is indeed soluble and of the correct size. The optimal growth temperature was found to be 28° C., as growth at higher temperatures (37° C.) was found to produce inclusion bodies. Sodium dithionite was added to reduce the cleared lysate, and upon addition of carbon monoxide the UV-vis spectra were collected and the results are shown in FIG. 7. A 450 nm absorbance, typical of a correctly folded and active P450 enzyme, was developed in the cleared cell lysate from E.coli cells expressing the 2E1-BM3 chimera (FIG. 7, thick line). A comparable level of absorbance at 450 nm is also visible in the positive control experiment where the BMP-FLD chimera was expressed as described in the previous section (FIG. 7, thin line). Moreover, negative control experiments on cell lysates of non-transformed E.coli did not show a peak at 450 nm under the same experimental conditions (FIG. 7, doffed line). These are consistent with the known fact that E.coli does not express endogenous P450 enzymes and the 2E1-BM3/1 chimera is indeed expressed in a folded, active form. These results indicate that the 2E1-BM3 chimera is indeed expressed in a soluble form in the cytosol of E.coli and it exhibits the fingerprint of an active P450 enzyme.

2E1-BM3/2

The second of the chimeras incorporates the cofactors haem, FAD and FMN more easily as can be appreciated by the uv-visible spectra of the oxidised reduced and reduced with CO shown in FIG. 10. Decreasing slightly in intensity. The shoulder in the 455-485 nm region no longer exists due to the reduction of the flavins. After carbon monoxide bubbling the Soret peak is shifted completely to 449 nm. In the inset the 500-600 nm region is enlarged. The pronouncement of the bands at 535 nm and 568 nm after-dithionite reduction is clearly seen; after formation of the protein/carbon monoxide complex the two bands are replaced by a broader band at ˜550 nm.

The most indicative peak is the transition at 450 nm for the haem reduced and complexed to CO, and the shoulders at 455-485 nm typical of FAD and FMN in the oxidised protein (these shoulders disappear in the reduced protein, as expected from the spectra of the reduced FAD and FMN). Also the difference spectra in FIG. 11, in which the arrows show the effect of increasing lauric acid concentration indicate that the chimera binds readily to this substrate. The results of the tests on oxygen consumption-in the presence of lauric acid for the 2E1-BM3/2 chimera show that it actively reacts with molecular oxygen, turning over the substrate into hydroxylated products. The results show Upon the first NADPH addition (75 nmol); 2.9 nmol oxygen consumed per minute in absence of substrate (63 nmol consumed in 22 minutes) and 3.8 nmol oxygen consumed per minute in presence of 500 mM lauric acid (62 nmol consumed in 16.5 minutes). Upon the second NADPH addition (75 nmol); 1.45 nmol oxygen consumed per minute in absence of substrate (58 nmol consumed in 40 minutes) and 1.8 nmol oxygen consumed per minute in presence of 500 mM lauric acid (55 nmol consumed in 33 minutes).

From these data and from the known structure of BM3 wild-type it is assumed that the FAD and FMN are bound to the BM3 which contributes the scaffold domain as the portions comprised in the chimera comprise the haem reductase portion. It is expected further that the haem is bound to the 2E1 component.

Conclusions

In conclusion, the feasibility of the molecular lego approach to the bacterial P450 BM3 and the human 2E1 has been demonstrated. An efficient electron transfer between two non-physiological partners, containing a P450 module was successfully achieved, and their gene-fused chimeric protein was successfully expressed in its active form. Also, the solubilisation of the human P450 2E1 was achieved, and its reduced form, in the presence of carbon monoxide, showed the typical absorption peak at 450 nm, characteristic of a folded P450 enzyme.

The results represent a step forward in constructing bio-molecular tools for the bio-analytical area, for example providing new P450 catalytic modules that can be used in artificial redox chains for future bioremediation, pharmacological and biosensing applications.

From the successful construction of the BM3-FLD chimera and its use in a method involving electron transfer to an electrode, and the successful formation of an active soluble mammalian GYP chimera with BM3 scaffold, it is believed that a chimera comprising the haem domain from a mammalian CYP, the scaffold portion from BM3 and electron transfer portion suitable for transfer of electrons to form an electrode may be possible to construct.

TABLE 4

Oligonucleotide primers used for the
construction of the BMP-FLD(n. 1 and
2) and the first 2E1-BM3 (n. 3-8)
chimeras.

| | | |
|---|---|---|
| n. | Code | Sequence |

| | | |
|---|---|---|
| 1 | P450 BM3 | CACAAGCAGCGGCATGTTATGAGCGTTTTC |
| 2 | FLD | AGGAAACAGCACATGCCTAAAGCTCTGATC |
| 3 | T7ProBMP | AATACGACTCACTATAGGGAGA |
| 4 | KpnIBMP | GACTTGATAGGTACCGCGTTAC |
| 5 | Kpnl2E1 | GCGCATGGGGTACCTGAGCGGCTACA |
| 6 | AvrII2E1 | CACACTCATGACCTAGGAATGAC |
| 7 | AvrIIBMR | CAGTCTGCTAAACCTAGGTCAAAAAAGGCAGAA |
| 8 | EcoRIBMR | TTATCCTAGCGAATTCTATACTTTTTTAGCCCACACG |

### REFERENCES

Barnes, H. J., M. P. Arlotto and M. R. Waterman (1991). “Expression and enzymatic activity of recombinant cytochrome P450 17alpha-hydroxylase in Escherichia coli.” Proceedings of the National Academy of Sciences 88: 5597-5601.

Connolly, M. L. (1983) Solvent-accessible surfaces of proteins and nucleic acids. Science, 221, 709-713.

Cosme, J. and E. F. Johnson (2000). “Engineering Microsomal Cytochrome P450 2C5 to be a soluble, monomeric enzyme.” The Journal of Biological Chemistry 275(4): 2545-2553.

Cuppvickery, J. R. and Poulos, T. (1995) Structure of P450eryF involved in erythromycin biosynthesis. Nature Struct. Biol. 2 (2), 144-153.

Darwish, K., Li, H., and Poulos, T. L. (1991) Engineering proteins, subcloning and hyperexpressing oxidoreductase genes. Prot. Engineering., 4, 701-708.

Doray, B., C. D. Chen and B. Kemper (1999). “Substitutions in the C-terminal portion of the catalytic domain partially reverse assembly defects introduced by mutations in the N-terminal linker sequence of cytochrome P450 2C2.” Biochemistry 38: 12180-12186.

Ellis, S. W., P. Y. Hayhurst, T. Lightfoot, G. Smith, J. Harlow, K. Rowland-yeo, C. Larsson, J. Mahling, C. K. Lim, C. R. Wolf, M. G. Blackburn, M. S. Lennard and G. T. Tucker (2000). “Evidence that serine 304 is not a key ligand-binding residue in the active site of cytochrome P450 2D6.” Biochemical Journal 345: 565-571.

Gilardi, G. Meharenna, Y. T., Tsotsou, G. E., Sadeghi, S. J., Fairhead, M and Giannini, S. (2002) “Molecular Lego: design of molecular assemblies of P450 enzymes for nanobiotechnology” Biosens. Bioelec. 17, 133-145.

Gillam, E. M. J., T. Baba, B. R. Kim, S. Ohmori and F. P. Guengerich (1993). “Expression of modified human cytochrome P450 3A4 in Escherichia coli and purification and reconstitution of the enzyme.” Archives of Biochemistry and Biophysics 305(1): 123-131.

Gillam, E. M. J., R. M. Wunsch, Y. F. Ueng, T. Schimada, P. E. B. Reily, T.

Kamtaki and F. P. Guengerich (1997). “Expression of cytochrome P4503A7 in Escherichia coli: Effects of 5′ modification and catalytic characterization of recombinant enzyme expressed in bicistronic format with NADPH-cytochrome P450 reductase.” Archives of Biochemistry and Biophysics 346(1): 81-90.

Gillam E. M. J., Guo Z., Guengerich F. P. (1994) Expression of modified human cytochrome P450 2E1 in Escherichia coli, purification, and spectral and catalytic properties. Arch. Biochem Biophys. 312, 59-66.

Guengerich, F. P. (1999) Cytochrome P450: regulation and role in drug metabolism, Annu. Rev. Pharmacol. Toxicol. 39, 1-17.

Hanna, I. H., J. R. Reed, F. P. Guengerich and P. F. Hollenberg (2000). “Expression of human cytochrome P450 2B6 in Escherichia coli: Characterization of catalytic activity and expression levels in human liver.” Archives of Biochemistry and Biophysics 376(1): 206-216.

Hasemann, C. A., Ravichandran, K. G., Peterson, J. A., and Deisenohofer, J. (1994) Crystal structure and refinement of cytochrome P450terp at 2.3 Å resolution. J Mol Biol 236, 1169-1185.

Hazzard, J. T., Govindaraj, S., Poulos, T. L. and Tollin, G. (1997) Electron transfer between the FMN and haem domains of cytochrome P450 BM3. J. Biol. Chem., 272, 7922-7926.

Heering, H. A., and Hagen, W. R. (1996) Complex electrochemistry of flavodoxin at carbon-based electrodes: results from a combination of direct electron transfer, flavin-mediated electron transfer and comproportionation. J. Electroanal. Chem., 404, 249-260.

Hosny, G., L. J. Roman, M. H. Mostafa and B. S. S. Masters (1999). “Unique properties of purified, Escherichia coli-expressed constitutive cytochrome P450 4A5.” Archives of Biochemistry and Biophysics 366(2): 199-206.

Jenkins, C. M., and Waterman, M. R. (1998) NADPH-flavodoxin reductase and flavodoxin from Escherichia coli as a soluble microsomal P450 reductase. Biochemistry, 37, 6106-6113.

Krey, G. D., Vanin, E. F. and Swenson, R. P. (1988) Cloning, nucleotide sequence and expression of the flavodoxin gene from D. vulgaris (Hildenborough). J. Biol. Chem., 263, 15436-15443.

Laskowski, R. A., MacArthur, M. W., Moss, D. S. and Thornton, J. M. (1993) PROCHECK: a programme to check the stereochemical quality of protein structure. J. Appl. Cryst. 26, 283-291.

Li, H. and Poulos, T. L., (1997) The structure of the cytochrome P450 BM-3 haem domain complexed with the fatty acid substrate, plamitoleic acid. Nature Str. Biol., 4, 140-146.

Li, H., Darwish, K. and Poulos, T. (1991) Characterisation of recombinant B.megaterium cytochrome P450 BM3 and its functional domains. J. Biol. Chem., 266, 11909-11914.

Licad-Coles, E., K. HE, H. Yin and M. A. Correia (1997). “Cytochrome P450 2C1 1: Escherichia coli expression, purification, functional characterization, and mechanism-based interaction of the enzyme.” Archives of Biochemistry and Biophysics 338(1): 35-42.

Lieber, C. S. (1997) Cytochrome P450 2E1: its physiological and pathological role. Phys. Rev. 77 (2), 518-538.

Narhi, L. O., and Fulco, A. J. (1986) Characterization of a catalytically self-sufficient 119,000-Dalton cytochrome P-450 monooxygenase induced by barbiturates in Bacillus megaterium. J. Biol. Chem., 261(16), 7160-7169.

Narhi, L. O., and Fulco, A. J. (1987) Identification and characterization of two functional domains in cytochrome P450 BM3, a catalytically self-sufficient monooxygenase induced by barbiturates in Bacillus megaterium. J. Biol. Chem., 262 (14), 6683-6690.

Nelson, D. R., and Strobel, H. W. (1998) On the membrane topology of vertebrate cytochrome P450 proteins. J. Biol. Chem., 263, 6038-6050.

Pernecky, S. J., Olken, N. M., Bestervelt, L. L. and Coon, M. J. (1995) Subcellular localisation, aggreagtion state and catalytic activity of microsomal P450 cytochromes modified in the N-terminal region and expressed in E.coli. Arch. Biochem. Biophys. 318 (2), 446-456.

Poulos, T. L. (1995) Cytochrome P450. Curr. Opin. Struct. Biol., 5, 767-774.

Poulos, T. L., Finzel, B. C., Howard, A. J. (1986) Crystal structure of substrate-free Pseudomonas putida cytochrome P450. J Am Chem Soc 25, 5314-5322.

Ravichandran, K. G., Boddupalli, S. S., Hasemann, C. A., Peterson, J. A., and Deisenhofer, J. (1993) Crystal structure of hemoprotein domain of P450BM-3, a prototype for microsomal P450s. Science, 261, 731-736.

Richardson, T. H., F. Jung, K. J. Griffin, M. Wester, J. L. Raucy, B. Kemper, L. M. Bomheim, C. Hassett, C. J. Omiencinski and E. F. Johnson (1995). “A universal approach to the expression of human & rabbit cytochrome P450's of the 2C subfamily in Escherichia coli.” Archives of Biochemistry and Biophysics 323(1): 87-96.

Ruettinger, R. T., Wen, L-P and Fulco, A. J. (1989) “Coding nucleotide, 5′ regulatory, and deduced amino acid sequences of P450 BM3, a single peptide cytochrome P450: NADPH-P450 reductase from Bacillus megaterium” J. Biol. Chem. 264 (19), 10987-10995.

Sadeghi, S. J., Meharenna, Y. T., and Gilardi, G. (1999) Flavodoxin as a module for transferring electrons to different c-type and P450 cytochromes in artificial redox chains. In: Ghisla, S., Kroneck, P., Macheroux, P., Sund, H. (Eds.), Flavins and flavoproteins. Agency for Scient. Publ., Berlin, pp. 163-166.

Sadeghi, S. J., Meharenna, Y. T., Fantuzzi, A., Valetti, F. and Gilardi, G. (2000a) Engineering artificial redox chains by molecular Lego, Faraday Discuss., 116, 135-153.

Sadeghi, S., Valetti, F., Cunha, C. A., Romao, M. J., Soares, C. M. and Gilardi, G. (2000b) Ionic strength dependence of the non-physiological electron transfer between flavodoxin and cytochrome c553 from D. vulgaris, J. Biol. Inorg. Chem. 5 (6), 730-737.

Sadeghi, S. J., Tsotsou, G. E., Fairhead, M., Meharenna, Y. T. and Gilardi, G. (2001) Rational design of P450 enzymes for biotechnology, In: Focus on Biotechnology. Physics and Chemistry Basis of Biotechnology. De Cuyper, M. and Bulte, J. (Eds), Kluwer Academic Publisher, in press.

Sagara, Y., H. J. Barnes and M. R. Waterman (1993). “Expression in Escherichia coli of functional cytochrome P450C17 lacking its hydrophobic amino-terminal signal anchor.” Archives of Biochemistry and Biophysics 306(1): 272-278.

Sandhu, P., T. Baba and F. P. Guengerich (1993). “Expression of modified cytochrome P4502C10 (2C9) in Escherichia coli, Purification and reconstitution of catalytic activity.” Archives of Biochemistry and Biophysics 306(2): 443-450.

Sandhu, P., Z. Guo, T. Baba, M. W. Martin, R. H. Tukey and F. P. Guengerich (1994). “Expression of modified human cytochrome P4501A2 in Escherichia coli: stabilization, purification, spectral characterization and catalytic activities of the enzyme.” Archives of Biochemistry an Biophysics 309(1): 168-177.

Schimada, T., R. M. Wunsch, I. H. Hanna, T. R. Sutter, F. P. Guengerich and E. M. J. Gillam (1998). “Recombinant human cytochrome P4501B1 expression in Escherichia coli.” Archives of Biochemistry and Biophysics 357(1): 111-120.

Schumyantsena, V. V., T. V. Bulko, N. N. Alexandrova Sokolov, R. D. Schmid, T. Bachmann and A. I. Archakov (1999). “N-terminal truncated cytochrome P450 2B4: catalytic activities and reduction with alternative electron sources.” Biochemical and Biophysical Research Communications. 263: 678-680.

Sevrioukova, I. F., Hazzard, J. T., Tollin, G., and Poulos, T. L. (1999) The FMN to Heme Electron Transfer in Cytochrome P450BM-3. J. Biol. Chem., 274(51), 36097-36106.

Shimoji, M., Yin, H., Higgins, L., Jones, J. P. (1998) Design of a novel P450: a functional bacterial-human cytochrome P450 chimera. Biochemistry 37, 848-52.

Soucek, P. (1999). “Expression of cytochrome P450 2A6 in Escherichia coli: Purification, spectral and catalytic characterization and preparation of polyclonal antibodies.” Archives of Biochemistry and Biophysics 370(2): 190-200.

Strobel, S. M. and J. R. Halpert (1997). “Reassessment of cytochrome P4502B2: Catalytic specificity and identification of four active site residues.” Biochemistry 36: 11697-11706.

Sueyosh, T., L. J. Park, R. Moore, R. V. Juvonen and M. Neigishi (1995). “Molecular engineering of microsomal P450 2a-4 to a stable, water-soluble enzyme.” Archives of Biochemistry and Biophysics 323(1): 265-271.

Szklarz, G. D., Y. A. HE and J. R. Halpert (1995). “Site-directed mutagenesis as a tool for molecular modeling of cytochrome P450 2B1.” Biochemistry 34(14312-14323).

Tsotsou, G. E., Cass, A. E. G. and Gilardi, G. (2002) High-throughput assay for cytochrome P450 BM3 for screening libraries of substrates and combinatorial mutants. Biosensors & Bioelectronics, 17, 119-130.

Umeno, M., McBride, W., Yang, .S., Gelboin, H. V., and Gonzalez, F. J. (1988) “Human ethanol-inducible P4502E1: complete gene sequence, promoter characterisation, chromosome mapping and cDNA-directed expression” Biochemistry, 27, 9006-9013.

Valetti, F., Sadeghi, S. J., Meharenna, Y., Gilardi, G. (1998) Engineering multi-domain redox proteins containing flavodoxin as bio-transformer: preparatory studies by rational design. Biosens. Bioelectron. 13, 675-685.

Watt, W., Tulinsky, A., Swenson, R. P., and Watenpaugh, K. D. (1991) Comparison of the crystal structures of a flavodoxin in its three oxidation states at cryogenic temperatures. J. Mol. Biol., 218, 195-208.

Williams, P. A., Cosme, J., Sridhar, V., Johnson, E. F., and McRee, D. E. (2000) Mammalian microsomal cytochrome P450 monooxygenase: Structural adaptations for membrane binding and functional diversity. Mol. Cell., 5, 121-131.

Woods, S. T., J. Sadleir, T. Downs, T. Triantopoulos, M. J. Headlam and R. C. Tuckey (1998). “Expression of catalytically active human cytochrome P450scc in Escherichia coli and mutagenesis of isoleucine-462.” Archives of Biochemistry and Biophysics 353(1): 109-115.

1. A water soluble chimeric protein which comprises a scaffold domain and a monooxygenase haem containing domain, in which the scaffold domain comprises an integral electron transfer domain capable of transferring electrons to the haem domains and is derived from BM3 and the haem domain is derived from a plant or an animal P450 enzyme.

2. A protein according to claim 1 in which the electron transfer domain is a flavo protein.

3. A protein according to claim 1 in which the haem domain is derived from a human P450 enzyme.

4. A protein according to claim 3 in which the haem domain is derived from human P450 2E1.

5. A protein according to claim 3 in which the haem domain comprises at least 200 contiguous residues of the wild-type P450 enzyme or an active mono oxygenase mutant thereof in which no more than 20 residues have been deleted or altered.

6. A protein according to claim 5 in which the haem domain comprises at least 250 continuous residues including the C-terminal or a sequence commending from no more than 50 residues in-board of the C-terminal of the wild-type P450 enzyme.

7. A protein according to claim 6 in which the haem domain comprises at least 300 residues.

8. A protein according to claim 4 in which no more than 10 residues have been deleted or altered relative to the wild-type P450 enzyme.

9-17. (canceled)

18. A protein according to claim 8 in which no more than 10 residues have been deleted or altered relative to the wild-type P450 enzyme.

19. A protein according to claim 1 in which the scaffold domain comprises at least 25 residues from the N terminal of wild-type BM3 protein or mutants thereof in which no more than 10 residues have been deleted or altered.

20. A protein according to claim 19 in which the scaffold domain comprises at least 50 residues from the N-terminal of wild-type BM3 protein and mutants thereof in which no more than 10 residues have been deleted or altered.

21. A protein according to claim 7 in which the scaffold domain comprises at least 50 residues from the N-terminal of wild-type BM3 protein and mutants thereof in which no more than 10 residues have been deleted or altered.

22. An oxidation process comprising the steps:
providing a water-soluble chimeric protein comprising a scaffold domain and a monooxygenase haem containing domain in which the scaffold domain comprises an integral electron transfer domain capable of transferring electrons to the haem domain is derived from BM3 and the haem domain is derived from a plant or an animal P450 enzyme and
contacting the chimeric protein with a substate in a reaction mixture in the prescence of oxygen whereby the substrate is oxidised to form an oxidised product and the electron transfer domain is converted to its oxidised form transferring an electron to the haem domain.

23. A process according to claim 22 in which the electron transfer domain is converted back to its reduced from by transfer of electrons from NAD(P)H or an electrode.

24. A process according to claim 22 in which the substrate is an analyte of interest and in which the extent of the oxidation reaction is measured whereby the presence or concentration of analyte is determined in the reaction mixture.

25. A process according to claim 24 in which transfer of an electron is from NAD(P)H and in which the extent of the oxidation reaction is determined by monitoring the production of NAD(P)+.

26. A process according to claim 24 in which transfer of an electron is from NAD(P)H, oxygen is present during the process and the extent of oxidation reaction is determined by monitoring oxygen consumption.

27. A microorganism transformed to synthesise a protein according to claim 1.

28. A microorganism transformed to synthesise a protein according to claim 6.

29. A microorganism transformed to synthesize a protein which comprises a scaffold domain and a monooxygenase haem containing domain, in which the scaffold domain comprises an integral electron transfer domain capable of transferring electrons to the haem domains and is derived from BM3 and the haem domain is derived from a plant or an animal P450 enzyme, transformed to synthesise a protein in which the scaffold domain comprises at least 50 residues from the N-terminal of wild-type BM3 protein and mutants thereof in which no more than 10 residues have been deleted or altered.

30. A microorganism according to claim 27 which is E. coli.

31. A plasmid comprising a gene capable of expressing a protein according to claim 1.


SEQUENCE LISTING (12 sequences)

SEQ ID NO: 1 - DNA, 30 bp, Artificial Sequence
  Description of Artificial Sequence oligonucleotide primer
cacaagcagcggcatgttatgagcgttttc

SEQ ID NO: 2 - DNA, 30 bp, Artificial Sequence
  Description of Artificial Sequence oligonucleotide primer
aggaaacagcacatgcctaaagctctgatc

SEQ ID NO: 3 - DNA, 22 bp, Artificial Sequence
  Description of Artificial Sequence oligonucleotide primer
aatacgactcactatagggaga

SEQ ID NO: 4 - DNA, 22 bp, Artificial Sequence
  Description of Artificial Sequence oligonucleotide primer
gacttgataggtaccgcgttac

SEQ ID NO: 5 - DNA, 26 bp, Artificial Sequence
  Description of Artificial Sequence oligonucleotide primer
gcgcatggggtacctgagcggctaca

SEQ ID NO: 6 - DNA, 23 bp, Artificial Sequence
  Description of Artificial Sequence oligonucleotide primer
cacactcatgacctaggaatgac

SEQ ID NO: 7 - DNA, 33 bp, Artificial Sequence
  Description of Artificial Sequence oligonucleotide primer
cagtctgctaaacctaggtcaaaaaaggcagaa

SEQ ID NO: 8 - DNA, 37 bp, Artificial Sequence
  Description of Artificial Sequence oligonucleotide primer
ttatcctagcgaattctatacttttttagcccacacg

SEQ ID NO: 9 - DNA, 1500 bp, Homo sapiens
atgtctgccctcggagtgaccgtggccctgctggtgtgggcggccttcctcctgctggtgtccatgtggaggcaggtgcacagcagctggaatctgcccccaggtcctttcccgcttcccatcatcgggaacctcttccagttggaattgaagaatattcccaagtccttcacccggttggcccagcgcttcgggccggtgttcacgctgtacgtgggctcgcagcgcatggtggtgatgcacggctacaaggcggtgaaggaagcgctgctggactacaaggacgagttctcgggcagaggcgacctccccgcgttccatgcgcacagggacaggggaatcatttttaataatggacctacctggaaggacatccggcggttttccctgaccaccctccggaactatgggatggggaaacagggcaatgagagccggatccagagggaggcccacttcctgctggaagcactcaggaagacccaaggccagcctttcgaccccaccttcctcatcggctgcgcgccctgcaacgtcatagccgacatcctcttccgcaagcattttgactacaatgatgagaagtttctaaggctgatgtatttgtttaatgagaacttccacctactcagcactccctggctccagctttacaataattttcccagctttctacactacttgcctggaagccacagaaaagtcataaaaaatgtggctgaagtaaaagagtatgtgtctgaaagggtgaaggagcaccatcaatctctggaccccaactgtccccgggacctcaccgactgcctgctcgtggaaatggagaaggaaaagcacagtgcagagcgcttgtacacaatggacggtatcaccgtgactgtggccgacctgttctttgcggggacagagaccaccagcacaactctgagatatgggctcctgattctcatgaaataccctgagatcgaagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccct

SEQ ID NO: 10 - protein, 493 aa, Homo sapiens
MSALGCTVALLVWAAFLLLVSMWRQVHSSWNLPPGPFPLPIIGNLFQLELKNIPKSFTRLAQRFGPVFTLYVGSQRMVVMHGYKAVKEALLDYKDEFSGRGDLPAFHAHRDRGIIFNNGPTWKDIRRFSLTTLRNYGMGKQGNESRIQREAHFLLEALRKTQGQPFDPTFLIGCAPCNVIADILFRKHFDYNDEKFLRLMYLFNENFHLLSTPWLQLYNNFPSFLHYLPGSHRKVIKNVAEVKEYVSERVKEHHQSLDPNCPRDLTDCLLVEMEKEKHSAERLYTMDGITVTVADLFFAGTETTSTTLRYGLLILMKYPEIEEKLHEEIDRVIGPSRIPAIKDRQEMPYMDAVVHEIQRFITLVPSNLPHEATRDTIFRGYLIPKGTVVVPTLDSVLYDNQEFPDPEKFKPEHFLNENGKFKYSDYFKPFSTGKRVCAGEGLARMELFLLLCAILQHFNLKPLVDPKDIDLSPIHIGFGCIPPRYKLCVIPRS

SEQ ID NO: 11 - DNA, 3225 bp, Artificial Sequence
  Chimeric human/bacterial construct
gaaattaatacgactcactatagggagaccacaacggtttccctctagaaacttaacaagtgaaggagggatcctatgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 12 - protein, 1049 aa, Artificial Sequence
  Chimeric human/bacterial construct
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMKNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGKEYPLEKGDELMVLIPQLHRDKTIWGDDVEEFRPEYFENPSAIPQHAFKPFGNGQRACIGQQFALHEATLVLGMMLKHFDFGDHTNYELDIKETLTLKPEGFVVKAKSKKIPLGGIPSPSTEQSAKKVRKKAENAHNTPLLVLYGSNMETAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFDLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPESEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG